(19) United States
(12) Patent Application Publication
(10) Pub. No.: US 2008/0260664 A1
(43) Pub. Date: Oct. 23, 2008

(54) Particles Functionalized with Organic Compounds

(76) Inventors: Thomas Walenzyk, Frankfurt (DE); Christophe Carola, Heidelberg (DE); Herwig Buchholz, Frankfurt (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/628,732

(22) PCT Filed: May 12, 2005
(86) PCT No.: PCT/EP2005/000517

§ 371 Date: Dec. 7, 2006

(30) Foreign Application Priority Data
Jun. 8, 2004 (EP) 04013515.4

(51) Int. Cl. A61K 8/02; A61Q 17/04

(52) U.S. Cl. 424/60; 424/59

(57) ABSTRACT

The present invention relates to particles comprising an inorganic network and organic compounds covalently bonded to the network via a spacer group, where the organic compounds are present in the interior of the particles and optionally also on the surface of the particles. The present invention furthermore relates to processes for the preparation of the particles and to the use thereof in formulations and compositions, in particular in compositions having light-protection properties.

The present invention relates to particles comprising an inorganic network and organic compounds covalently bonded to the network via a spacer group, where the organic compounds are present in the interior of the particles and optionally also on the surface of the particles. The present invention furthermore relates to processes for the preparation of the particles and to the use thereof in formulations and compositions, in particular in compositions having light-protection properties.

Organic compounds play a major role in connection with cosmetic or dermatological formulations and compositions. Thus, for example, organic compounds from a very wide variety of classes of compound are employed as UV filters in order to protect the skin against the effects of sunlight and associated undesired side effects, such as, for example, skin ageing or wrinkling. The common UV filters usually act by forming a film on the surface of the skin. The UV filters absorb certain regions of sunlight, so that the filtered radiation is not able to penetrate into relatively deep layers of the skin. At the same time, however, it is increasingly being attempted also to suppress the penetration of the UV filters into the skin in order to prevent possible skin damage or allergy-triggering processes.

Thus, EP 1 205 177 proposes employing a conjugate comprising an organic pigment and an active ingredient based on organic compounds, for example a UV filter, which is covalently bonded to the inorganic pigment. For the preparation of this conjugate, correspondingly functionalised organic compounds are bonded to the surface of the pigments. The UV filter/pigment systems obtained in this way cannot penetrate into the skin owing to their particulate character. However, contact of the skin with the compounds applied to the surface of the pigments is not completely prevented in this way since the compounds are able to interact with the skin on the surface. Furthermore, the maximum proportion of organic compounds to be applied is limited by the surface area of the pigment, i.e. the maximum active ingredient concentration correlates directly with the available surface area. Precisely in the area of UV filters, the highest possible proportion of active ingredient is particularly important in order to achieve the maximum protective effect, i.e. in order to ensure optimum protection against solar radiation. There is therefore an urgent demand for active ingredient/carrier systems which on the one hand have only a low tendency to penetrate into the skin and on the other hand have the highest possible active ingredient content.

The present invention accordingly had the object of providing materials which enable the advantageous effects, preferably light-absorbing effects, of organic compounds easier to utilise without penetration of the organic compounds into the skin being necessary.

This object is achieved by the particles in accordance with the present invention. The present invention accordingly relates to particles comprising an inorganic network and organic compounds covalently bonded to the network via a spacer group, where the organic compounds are present in the interior of the particles and optionally also on the surface of the particles. The present invention furthermore relates to processes for the preparation of the particles according to the invention, comprising
a) the reaction of an organic compound with formation of a covalent bond to a substance having at least two reactive groups separated by a spacer group and
b) the copolymerisation of the compound obtained in a) with a precursor which is suitable for the construction of an inorganic network.

The present invention likewise relates to the use of the particles according to the invention in formulations and compositions and to compositions comprising the particles according to the invention.

The particles in accordance with the present invention have the advantage that the binding of the organic compounds into the core of the particles minimises the contact, for example, with the skin. In addition, the relatively large volume of the particles in relation to the surface area enables more active ingredients to be integrated into the particles in a majority of the suitable particles than would be possible with pure application to the surface. A further advantage is that the binding of the organic compounds into the core of the particles prevents preparation problems in the preparation of formulations which arise due to interaction of individual composition constituents with one another, such as crystallisation processes, precipitations and agglomeration. In addition, the photostability of the organic compounds employed, in particular in the case of UV filters, can increase if they are embedded in the core of a matrix, as in the case of the present invention.

The particles according to the invention comprise an inorganic network and organic compounds covalently bonded to the network via a spacer group, where the compounds are present in the core of the particles and optionally also on the surface of the particles. The shape of the particles is not crucial per se, the particles are preferably spherical. Non-functionalised spherical particles can be prepared particularly simply and exhibit, for example on application to the skin, a particularly good skin feel. By contrast, the preparation of spherical particles functionalised on the surface proves to be difficult since the functionalisation may cause crosslinking of the forming particles, which makes the formation of spheres more difficult or prevents it completely. The present invention therefore allows the preparation of spherical particles with functionalisation present in the core of the particles which combines the advantageous properties of spheres, for example better skin feel, with the advantageous properties of organic compounds, for example UV protection. The size, in the simplest case the maximum diameter, of the particles can vary between 1 nm and 250 μm, preferably in the range from 1 nm to 1 μm and very particularly preferably between 30 nm and 700 nm.

The inorganic network of the particles according to the invention can be, for example, an oxide, oxide hydrate, phosphate, carbonate, sulfate or nitride, where the oxide, oxide hydrate, phosphate, carbonate, sulfate or nitride of a metal, semimetal or non-metal can be present, preferably an oxide of a metal or semimetal. Corresponding oxides are, for example, magnesium oxide, aluminium oxide, silicon oxide, zinc oxide, cerium oxide, titanium oxide, zirconium oxide, manganese oxide, boron oxide, iron oxide or a mixture thereof. Examples of phosphates, carbonates, sulfates or nitrides are, inter alia, magnesium carbonate, calcium carbonate, barium sulfate, calcium sulfate, calcium phosphate, boron nitride, where the substances mentioned may be present individually or in the form of a mixture. The inorganic network is preferably silicon oxide, in particular silicon dioxide. Particles, in particular spherical particles, comprising silicon oxide as inorganic network are readily accessible and prove to be particularly suitable in dermatological applications, since they do not react with the skin, are very substantially chemically inert and are thus toxicologically acceptable. In the case of spherical SiO₂ particles, a good skin feel can be produced in the case of application in topical compositions, which may be attributed to the spherical character of the particles.

The organic compounds covalently bonded to the network can be selected from the group of light-absorbent organic compounds, compounds having antioxidant and/or free-radical-inhibiting properties, repellents, preservatives and/or derivatives of these compounds, preferably light-absorbent compounds.

The light-absorbent compounds can be selected from the group of dibenzoylmethane derivatives, aminobenzoic acid, cinnamic acid, salicylic acid, benzylidenecamphor, phenylbenzimidazole, diphenyl acrylate, triazine, benzophenone, diarylbutadiene, vinyl-containing amides and/or derivatives thereof which are described, for example, in the application WO 93/04665. Further examples of organic filters are indicated in patent application EP-A 0 487 404.

The dibenzoylmethane derivatives which can be used in accordance with the invention can be selected, in particular, from the dibenzoylmethane derivatives of the following formula:

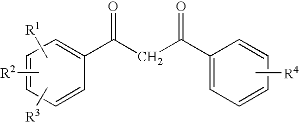

in which R¹, R², R³ and R⁴, which are identical or different from one another, denote hydrogen, a straight-chain or branched C₁₋₈-alkyl group or a straight-chain or branched C₁₋₈-alkoxy group. In accordance with the present invention, it is of course possible to use one dibenzoylmethane derivative or a plurality of dibenzoylmethane derivatives. Of the dibenzoymethane derivatives to which the present invention more specifically relates, mention may be made, in particular, of:
-2-methyldibenzoylmethane,
-4-methyldibenzoylmethane,
-4-isopropyidibenzoylmethane,
-4-tert-butyidibenzoylmethane,
-2,4-dimethyldibenzoylmethane,
-2,5-dimethyidibenzoylmethane,
-4,4′-diisopropyldibenzoylmethane,
-4,4′-methoxy-tert-butyidibenzoylmethane,
-2-methyl-5-isopropyl-4′-methoxydibenzoylmethane,
-2-methyl-5-tert-butyl-4′-methoxydibenzoylmethane,
-2,4-dimethyl4′-methoxydibenzoylmethane and
-2,6-dimethyl4-tert-butyl4′-methoxydibenzoylmethane, this list being non-restrictive. Of the above-mentioned dibenzoylmethane derivatives, particular preference is given in accordance with the invention to 4,4′-methoxy-tert-butyidibenzoylmethane and especially 4,4′-methoxy-tert-butyldibenzoylmethane, which is commercially available under the trade name Eusolex® 9020 from Merck KGaA, where this filter conforms to the following structural formula:

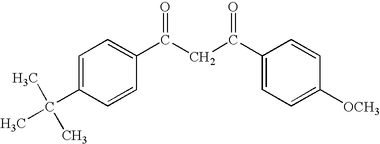

A further dibenzoylmethane derivative which is preferred in accordance with the invention is 4-isopropyldibenzoylmethane.

The light-absorbent compounds can furthermore be benzophenone or derivatives of benzophenone, such as particularly preferably 2-hydroxy4-methoxybenzophenone (for example Eusolex® 4360) or 2-hydroxy-4-meth-oxybenzophenone-5-sulfonic acid and its sodium salt (for example Uvinul® MS40). Further examples of suitable light-absorbent substances include benzylidenecamphor derivatives, such as 3-(4′-methylbenzylidene)-dl-camphor (for example Eusolex® 6300), 3-benzylidenecamphor (for example Mexoryl® SD), polymers of N-{(2 and 4)-[(2-oxoborn-3-ylidene)methyl]-benzyl}acrylamide (for example Mexoryl® SW), N,N,N-trimethyl4-(2-oxo-born-3-ylidenemethyl)anilinium methylsulfate (for example Mexoryl® SK) or (2-oxoborn-3-ylidene)toluene-4-sulfonic acid (for example Mexoryl® SL), methoxycinnamic acid esters, such as octyl methoxycinnamate (for example Eusolex® 2292), isopentyl 4-methoxycinnamate, for example as a mixture of the isomers (for example Neo Heliopane E 1000), salicylate derivatives, such as 2-ethylhexyl salicylate (for example Eusolex® OS), 4-isopropylbenzyl salicylate (for example Megasol®) or 3,3,5-trimethylcyclohexyl salicylate (for example Eusolex® HMS), 4-aminobenzoic acid and derivatives, such as 4-aminobenzoic acid, 2-ethylhexyl 4-(dimethylamino)benzoate (for example Eusolex® 6007), ethoxylated ethyl 4-aminobenzoate (for example Uvinul® P25), phenylbenzimidazolesulfonic acids, such as 2-phenylbenzimidazole-5-sulfonic acid and the potassium, sodium and triethanolamine salts thereof (for example Eusolex® 232), 2,2-(1,4-phenylene)bisbenzimidazole4,6-disulfonic acid and salts thereof (for example Neoheliopan® AP) or 2,2-(1,4-phenylene)bisbenzimidazole-6-sulfonic acid, and further substances, such as
-2-ethylhexyl 2-cyano-3,3-diphenylacrylate (for example Eusolex® OCR),
-3,3′-(1,4-phenylenedimethylene)bis-7,7-dimethyl-2-oxobicyclo[2.2.1]hept-1-ylmethanesulfonic acid and salts thereof (for example Mexoryl® SX) and
-2,4,6-trianilino-(p-carbo-2′-ethylhexyl-1′-oxy)-1,3,5-triazine (for example Uvinul® T 150)
hexyl 2-(4-diethylamino-2-hydroxybenzoyl)benzoate (for example Uvinul®UVA Plus, BASF).

Further suitable organic UV filters are, for example,
2-(2H-benzotriazol-2-yl)-4-methyl-6-(2-methyl-3-(1,3,3,3-tetramethyl-1 -(trimethylsilyloxy)disiloxanyl)propyl)phenol (for example Silatrizole®),
2-ethylhexyl 4,4′-[(6-[4-((1,1-dimethylethyl)aminocarbonyl)phenylamino]-1,3,5-triazine-2,4-diyl)diimino]bis(benzoate) (for example Uvasorb® HEB),
α-(trimethylsilyl)-ω-[trimethylsilyl)oxy]poly[oxy(dimethyl [and approx. 6% of methyl[2-[p-[2,2-bis(ethoxycarbonyl]vinyl]phenoxy]-1-methyleneethyl] and approx. 1.5% of methyl[3-[p-[2,2-bis(ethoxycarbonyl)vinyl)phenoxy)-propenyl) and 0.1 to 0.4% of (methylhydrogen]silylene]] (n≈60) (CAS No. 207 574-74-1)
2,2′-methylenebis(6-(2H-benzotriazol-2-yl)4-(1,1,3,3-tetramethylbutyl)-phenol) (CAS No. 103 59745-1)
2,2′-(1,4-phenylene)bis(1H-benzimidazole-4,6-disulfonic acid, mono-sodium salt) (CAS No. 180 898-37-7) and
2,4-bis{[4-(2-ethylhexyloxy)-2-hydroxy]phenyl}-6-(4-methoxyphenyl)-1,3,5-triazine (CAS No.103 597-45-, 187 393-00-6).
-2-ethylhexyl 4,4′-[(6-[4-((1,1-dimethylethyl)aminocarbonyl)phenylamino]-1,3,5-triazine-2,4-diyl)diimino]bis(benzoate) (for example Uvasorb® HEB),

Preferred compounds having UV-filtering properties are 3-(4′-methylbenzylidene)-dl-camphor, 1-(4-tert-butylphenyl)-3-(4-methoxyphenyl)propane-1,3-dione, 4-isopropyldibenzoylmethane, 2-hydroxy-4-methoxybenzophenone, octyl methoxycinnamate, 3,3,5-trimethylcyclohexyl salicylate, 2-ethylhexyl 4-(dimethylamino)benzoate, 2-ethylhexyl 2-cyano-3,3-diphenylacrylate, 2-phenylbenzimidazole-5-sulfonic acid and the potassium, sodium and triethanolamine salts thereof.

Very particular preference is given to the use of 2-hydroxy-4-methoxy-benzophenone and/or 4,4′-methoxy-tert-butyldibenzoylmethane.

Suitable substances having antioxidant and/or free-radical-inhibiting properties include, for example, flavonoids, coumarones, amino acids (for example glycine, histidine, tyrosine, tryptophan) and derivatives thereof, imidazoles (for example urocanic acid) and derivatives thereof, peptides, such as, for example, D,L-carnosine, D-carnosine, L-carnosine and derivatives thereof (for example anserine), carotinoids, carotenes (for example α-carotene, β-carotene, lycopene) and derivatives thereof, chlorogonic acid and derivatives thereof, lipoic acid and derivatives thereof (for example dihydrolipoic acid), aurothioglucose, propylthiouracil and other thiols (for example thioredoxin, glutathione, cysteine, cystine, cystamine and the glycosyl, N-acyl, methyl, ethyl, propyl, amyl, butyl and lauryl, palmitoyl, oleyl, linoleyl, cholesteryl and glyceryl esters thereof, dilauryl thiodipropionate, distearyl thiodipropionate, thiodipropionic acid and derivatives thereof (esters, ethers, peptides, lipids, nucleotides and nucleosides), and sulfoximine compounds (for example buthionine sulfoximines, homo-cysteine sulfoximine, buthionine sulfones, penta-, hexa- and heptathionine sulfoximine), chelating agents (for example α-hydroxy fatty acids, palmitic acid, phytic acid, lactoferrin), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof, vitamin C and derivatives (for example ascorbyl palmitate, ascorbyl acetate), tocopherols and derivatives (for example vitamin E acetate), vitamin A and derivatives (for example vitamin A palmitate), and coniferyl benzoate of benzoin resin, rutinic acid and derivatives thereof, α-glycosylrutin, ferulic acid, furfurylideneglucitol, carnosine, butylhydroxy-toluene, butylhydroxyanisole, nordihydroguaiaretic acid, trihydroxybutyrophenone, quercetin, uric acid and derivatives thereof, mannose and derivatives thereof, vitamin E and derivatives thereof, stilbenes and derivatives thereof (for example stilbene oxide, trans-stilbene oxide), and BHT (2,6-di-tert-butyl-4-methylphenol).

Preferred antioxidants include flavonoids, coumaranones, vitamins and BHT.

Flavonoids are taken to mean the glycosides of flavonones, flavones, 3-hydroxyflavones (=flavonols), aurones, isoflavones and rotenoids (Rompp Chemie Lexikon [Römpp's Lexicon of Chemistry], Volume 9, 1993). For the purposes of the present invention, however, this term is also taken to mean the aglycones, i.e. the sugar-free constituents, and the derivatives of the flavonoids and aglycones. For the purposes of the present invention, the term coumaranones is also taken to mean derivatives thereof.

Preferred flavonoids are derived from flavonones, flavones, 3-hydroxy-5 flavones, aurones and isoflavones, in particular from flavonones, flavones, 3-hydroxyflavones and aurones. The flavonoids and coumaranones are preferably selected from the compounds of the formula (1):

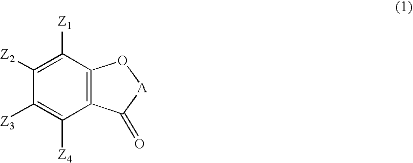

in which:

Z₁ to Z₄ each, independently of one another, denote H, OH, alkoxy (OR), hydroxyalkoxy, mono- or oligoglycoside radicals, where the alkoxy and hydroxyalkoxy groups may be branched and unbranched and may have 1 to 18 carbon atoms and where sulfate or phosphate may also be bonded to the hydroxyl groups of the said radicals.

A is selected from the group consisting of the sub-formulae (1A), (1B) and (1C)

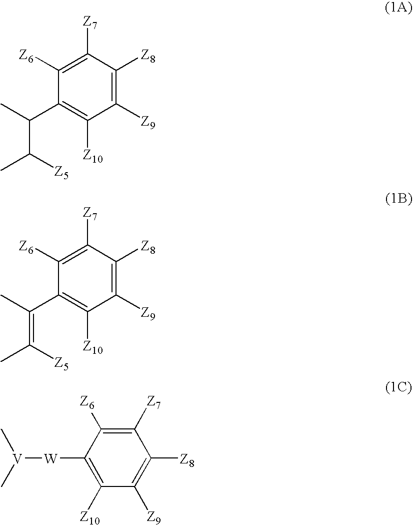

where Z₅ denotes H, OH or OR,

Z₆ to Z₁₀ have the meaning of the radicals Z₁ to Z₄, and

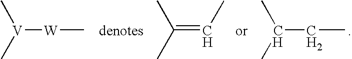

The alkoxy groups are preferably linear and have 1 to 12, preferably 1 to 8, carbon atoms. These groups thus conform to the formula —O—(CH₂)ₘ-H, where m denotes 1,2,3,4,5,6,7 or 8 and in particular 1 to 5.

The hydroxyalkoxy groups are preferably linear and have 2 to 12, preferably 2 to 8, carbon atoms. These groups thus conform to the formula —O—(CH₂)ₙ-OH, where n denotes 2,3,4,5,6,7 or 8, in particular 2 to 5 and particularly preferably 2.

The mono- and oligoglycoside radicals are preferably built up from 1 to 3 glycoside units. These units are preferably selected from the group of the hexosyl radicals, in particular the rhamnosyl radicals and glucosyl radicals. However, other hexosyl radicals, for example allosyl, altrosyl, galactosyl, gulosyl, idosyl, mannosyl and talosyl, may also advantageously be used if appropriate. It may also be advantageous in accordance with the invention to use pentosyl radicals.

In a preferred embodiment,
Z₁ and Z₃ have the meaning H,
Z₂ and Z₄ have a meaning other than H, in particular they denote OH, methoxy, ethoxy or 2-hydroxyethoxy,
Z₅ has the meaning H, OH or a glycoside radical which is built up from 1 to 3, preferably 1 or 2, glycoside units,
Z₆, Z₉ and Z₁₀ have the meaning H, and
Z₇ and Z₈ have a meaning other than H, in particular they denote OH, methoxy, ethoxy or 2-hydroxyethoxy.

In a further preferred embodiment, the flavonoids are selected from the following compounds: 4,6,3′,4′-tetrahydroxyaurone, quercetin, rutin, isoquercetin, anthocyanidine (cyanidine), eriodictyol, taxifolin, luteolin, trishydroxyethylquercetin (troxequercetin), trishydroxyethylrutin (troxerutin), trishydroxyethylisoquercetin (troxeisoquercetin), trishydroxyethylluteolin (troxeluteolin). Of the flavonoids, particular preference is given to rutin and troxerutin. Troxerutin is especially preferred. Of the coumaranones, 4,6,3′,4′-tetrahydroxybenzylcoumaranone-3 is preferred. Further suitable antioxidants are mentioned below or are accessible to the person skilled in the art without inventive step.

Suitable repellents include amides and derivatives thereof, in particular N,N-diethyl-3-methylbenzamide, ethyl 3-[N-n-butyl-N-acetyl]aminopropionate (IR3535®) and N,N-diethyl caprylamide (IR790®).

Suitable preservatives include benzalkonium chloride, benzoic acid and salts thereof (such as, for example, sodium benzoate), methylparaben, ethylparaben, propylparaben, sorbic acid and salts thereof (such as, for example, potassium sorbate), cetyl pyridinium chloride, cetrimonium chloride and salicylic acid and salts thereof (such as, for example, sodium salicylate).

The spacer group which links the inorganic network to the organic compound has the general formula

(-A)ₐMe(R¹)_{b}(R²)_{c}-X—

where
A=O, S, NH
Me=Si, Al, Ti, Zr
R¹ and R² may be identical or different and denote a straight-chain or branched alkyl, alkoxy, halogen or hydroxyl group a is 1 to 3
b and c may be 0-2, with the condition that a+b+c=3 for Me=Si, Ti, Zr and a+b+c=2 for Me=Al, and
X=a straight-chain or branched alkyl group having up to 20, preferably having 1 to 12 and particularly preferably having 3 to 12, carbon atoms, and where the spacer group is bonded to the inorganic network via the substituent (-A)ₐ.

Depending on the number of substituents (-A), one or more covalent bonds to the inorganic network may be present via the central atom Me of the spacer group. The central atom Me is preferably silicon. Spacer groups of this type are particularly suitable for linking organic compounds to the inorganic network.

The straight-chain or branched C₁-C₂₀-alkyl group is, for example, methyl, ethyl, isopropyl, propyl, butyl, sec-butyl or tert-butyl, furthermore also pentyl, 1-, 2- or 3-methylbutyl, 1,1-, 1,2- or 2,2-dimethylpropyl, 1-ethylpropyl, hexyl, heptyl, octyl, nonyl, decyl, undecyl, dodecyl, tridecyl or tetradecyl, optionally perfluorinated alkyl groups, for example difluoromethyl, trifluoromethyl, pentafluoroethyl, heptafluoropropyl or nonafluorobutyl. The straight-chain or branched alkyl group is preferably isopropyl, propyl or butyl.

The straight-chain or branched alkoxy group is, for example, methoxy, ethoxy, propoxy, butoxy, pentoxy, isopropoxy, preferably methoxy or ethoxy.

Halogen is F, Cl, Br, I, preferably Cl or Br.

In a further embodiment of the present invention, the surface of the particles according to the invention may be additionally functionalised. For functionalisation of the particles, organic compounds are preferably covalently bonded to the surface. The organic compounds can be selected from the group of light-absorbent organic compounds, compounds having antioxidant and/or free-radical-inhibiting properties, repellents, preservatives and/or derivatives of these compounds. Examples of the above-mentioned compounds have been mentioned above in the description of the particles according to the invention. The proportion of the organic compounds and thus, for example, of the active ingredients can be further increased in this way. In the case of, for example, UV filters, the proportion of absorbent substance per particle can be increased in order to enable the protective action of the UV filters to be enhanced. The surface of the particles can be functionalised with the identical organic compounds present in the interior of the particles and/or with organic compounds which are different there-from. A combination of different active ingredients can thus also be achieved, i.e., for example, a UV filter is present in the interior of the particles and compounds having antioxidant properties or repellents are located on the surface. There are no restrictions for the individual combinations, i.e. all active ingredients can be combined with one another so that multi-functional particles are obtained.

The present invention likewise relates to processes for the preparation of the particles according to the invention, comprising
a) the reaction of an organic compound with formation of a covalent bond to a substance having at least two reactive groups separated by a spacer group and
b) the copolymerisation of the compound obtained in a) with a precursor which is suitable for the construction of an inorganic network.

In the first step of the process according to the invention, an organic compound is reacted with a substance having at least two reactive groups separated by a spacer group in such a way that the organic compound is covalently bonded to the substance. Substances having at least two reactive groups separated by a spacer group that can be employed are those of the general formula

(R³A)ₐMe(R¹)_{b}(R²)_{c}-X—Y

where
A=O, S, NH
Me=Si, Al, Ti, Zr
R¹ and R² may be identical or different and denote a straight-chain or branched alkyl, alkoxy, halogen or hydroxyl group
R³=hydrogen or a straight-chain or branched alkyl group
a=1 to 3
b and c may be 0-2, with the condition that a+b+c =3 for Me=Si, Ti, Zr and a+b+c=2 for Me=Al, and
X=a straight-chain or branched alkyl group having up to 20, preferably having 1 to 12 and particularly preferably having 3 to 12, carbon atoms, and
Y=straight-chain or branched alkenyl, alkynyl, alkoxy, SH, NH₂, carboxyl, halogen or hydroxyl, where the covalent bond is formed between the organic compound and the spacer group with cleavage or via linking to the reactive group Y. Generally suitable for the linking are all types of reaction for the formation of covalent bonds that are known to the person skilled in the art, such as, for example, addition, substitution, hydrosilylation, etc. The choice of suitable linking reactions is subject to the expert knowledge of the person skilled in the art and is given, for example, in EP 1 205 177, the disclosure content of which is incorporated herein by way of reference.

A straight-chain or branched alkenyl having 2 to 20 C atoms, where a plurality of double bonds may also be present, is, for example, vinyl, allyl, 2- or 3-butenyl, isobutenyl, sec-butenyl, furthermore 4-pentenyl, iso-pentenyl, hexenyl, heptenyl, octenyl, —C₉H₁₇, —C₁₀H₁₉ to —C₂₀H₃₉; preferably vinyl, allyl, 2- or 3-butenyl, isobutenyl, sec-butenyl, preference is furthermore given to 4-pentenyl, isopentenyl or hexenyl.

A straight-chain or branched alkynyl having 2 to 20 C atoms, where a plurality of triple bonds may also be present, is, for example, ethynyl, 1- or 2-propynyl, 2- or 3-butynyl, furthermore 4-pentynyl, 3-pentynyl, hexynyl, heptynyl, octynyl, —C₉H₁₅, —C₁₀H₁₇ to —C₂₀H₃₇, preferably ethynyl, 1- or 2-propynyl, 2- or 3-butynyl, 4-pentynyl, 3-pentynyl or hexynyl.

The organic compounds which are linked to the spacer group can be selected from the group of light-absorbent organic compounds, compounds having antioxidant and/or free-radical-inhibiting properties, repellents, preservatives and/or derivatives of these compounds. Examples thereof have already been mentioned.

The second step of the processes according to the invention involves the copolymerisation of the compound obtained in the first step with a precursor which is suitable for the construction of an inorganic network. The second step is preferably a hydrolytic polycondensation. In this way, a multiplicity of inorganic networks can be built up, it being possible to adapt the reaction conditions in a variety of ways. Processes of this type are known and are given, for example, in the publications by W. STÖBER et al. in J. Colloid and Interface Science 26, 62 (1968) and 30, 568 (1969) and U.S. Pat. No. 3,634,588, which reveal the basic reaction conditions for this purpose. These propose that, for example, tetraalkoxysilane is introduced into an excess of an aqueous-alcoholic-ammoniacal hydrolysis mixture, with intensive mixing being ensured by suitable measures, such as stirring, shaking or ultrasound treatment. Depending on the choice of the specific experimental parameters, SiO₂ particles having different average particle size and varying particle-size distribution can be obtained here. This and analogous processes are suitable for the preparation of the inorganic networks in accordance with the present invention.

Precursors which can be employed for the construction of the inorganic network are, for example, compounds of the elements magnesium, aluminium, silicon, zinc, cerium, titanium, zirconium, manganese, boron and iron which contain hydroxides, halides, alkoxides or other hydrolysable groups, and/or mixtures thereof. The precursors are preferably alkoxides of silicon, such as, for example, tetraalkoxysilanes. Suitable tetraalkoxysilanes which can be employed are all readily hydrolysable silicic acid orthoesters of aliphatic alcohols. Primarily suitable here are the esters of aliphatic alcohols having 1 to 5 C atoms, i.e. the alkoxy group can be a methoxy, ethoxy, propbxy, butoxy or pentoxy group, where the respective isomers are taken into account. The above-mentioned tetraalkoxysilanes can be employed individually, but also in the form of a mixture. Preference is given to the use of the silicic acid orthoesters of C₁-C₃-alcohols, in particular tetraethoxysilane. Also suitable, however, are organotrialkoxysilanes, as are known, for example, for the modification of silica gels. The organo groups in these compounds can be aliphatic radicals having 1-20 C atoms, optionally functionalised, for example by hydroxyl, thio, amino or carboxyl groups, or halogen and alkenyl radicals. The incorporation of functionalised organo groups into the SiO₂ matrix of the particles additionally facilitates later further modification by the formation of covalent bonds in a known manner. Examples of organotrialkoxysilanes of this type are, for example, methyltriethoxysilane, ethytriethoxysilane, hexyltriethoxysilane, octytriethoxysilane, dodecyltriethoxysilane, octadecyltriethoxysilane, vinyltriethoxysilane, 3-hydroxypropyltriethoxysilane, 3-chloropropyltriethoxy-silane 3-aminopropyltriethoxysilane, 3-glycidoxypropyltriethoxysilane, 3-mercaptopropyltriethoxysilane, 3-isothiocyanatopropyltriethoxysilane, 3-(aminoethylamino)propyltriethoxysilane, 3-methacryloxypropyltriethoxysilane, 3-acetoxypropyltriethoxysilane, N-(3-triethoxysilylpropyl)-N′-(1-5 phenyl-1 -hydroxyisopropyl)thiourea, N-(3-triethoxysilylpropyl)-N′-(1-phenylethyl)thiourea or mixtures thereof.

In the simplest embodiment of the processes according to the invention, the organic compounds are firstly reacted with the substance having at least two reactive groups separated by a spacer group, and the resultant functionalised compound is isolated. Corresponding synthetic procedures are given, for example, in EP 1 205 177. In the following step, a corresponding precursor and the compound obtained in the first step are introduced together or individually into a copolymerisation-initiating mixture, preferably a hydrolysis mixture, where the precursor and the compound in the case of individual addition can be added simultaneously or successively. The addition is preferably carried out in the form of a mixture of the precursor and the functionalised compound. The copolymerisation-initiating mixture may already comprise all components necessary for initiation of the polymerisation, but individual constituents may also be added successively. In the case of the hydrolytic polycondensation that is preferably employed, the hydrolysis mixture is preferably in the form of an ethanol/water/-ammonia mixture, into which the precursor and the functionalised compound are introduced with initiation of the polycondensation. Further variants of the process procedure are subject to the expert knowledge of the person skilled in the art and can be derived simply. The reaction can be carried out at temperatures in the range from 5 to 80° C., preferably in the range from 20 to 80° C. and particularly preferably at 25 to 65° C. The particles obtained can be isolated by methods familiar to the person skilled in the art, for example by washing, extraction or centrifugation. The particles according to the invention are preferably isolated by extraction and/or centrifugation.

In a further process according to the invention, the surface of the particles is additionally functionalised. To this end, organic compounds are preferably covalently bonded to the surface of the particles, where the organic compounds are preferably selected from the group of light-absorbent organic compounds, compounds having antioxidant and/or free-radical-inhibiting properties, repellents, preservatives and/or derivatives of these compounds. Examples of suitable compounds of this type have already been described above.

The additional surface functionalisation is ideally carried out by reaction of the particles according to the invention with the corresponding organic compounds, preferably with the functionalised compounds obtained in process step a) of the process according to the invention. The functionalised compounds have adequate reactivity in order to be able to react with the surface of the particles according to the invention. Processes for subsequent functionalisation are known to the person skilled in the art, examples are given, inter alia, in EP 1 205 177. In the simplest case, for example in the case of hydrolytic polycondensation, the particles according to the invention are isolated and re-reacted with the functionalised compounds under the hydrolysis conditions already described. This additional process step can be carried out after isolation of the particles according to the invention, alternatively the functionalisation can also be carried out directly after the synthesis of the particles according to the invention. This procedure is preferred. In this way, the individual process steps can be combined with one another, which enables a significant simplification of the process procedure and a saving of starting materials required, for example in the form of the hydrolysis mixture. The additional functionalisation can optionally be based on the reaction of the particles formed with still unreacted fractions of the compounds employed in the preparation of the particles. Alternatively, it is also possible to achieve the functionalisation by additional addition of further fractions of the organic compounds, in particular if the most complete functionalisation possible of the surface of the particles is desired.

The particles according to the invention are basically suitable for use in any form of formulation or composition, such as, for example, cosmetic compositions, but also in compositions which can be employed in the industrial sector, such as, for example, paints or coatings. The particles according to the invention are preferably employed in topical compositions, in particular in cosmetics. Besides the particles, the compositions according to the invention preferably comprise at least one cosmetically, pharmaceutically and/or dermatologically tolerated carrier and/or adjuvant. The particles according to the invention preferably allow the preparation of compositions having light-protection properties for a multiplicity of application variants and media, where the light-protection properties can be selected individually through the suitable choice of the organic compound, in this case the UV filters.

Preferred compositions having light-protection properties additionally comprise at least one further organic and/or inorganic UV filter, preferably a dibenzoylmethane derivative. The dibenzoylmethane derivatives used for the purposes of the present invention are products which are already well known per se and are described, in particular, in the specifications FR-A-2 326 405, FR-A-2 440 933 and EP-A-0 114 607.

The dibenzoylmethane derivatives which can be used in accordance with the invention may be selected, in particular, from the dibenzoylmethane derivatives of the following formula:

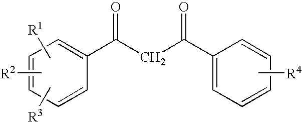

in which R¹, R², R³ and R⁴, which are identical or different from one another, denote hydrogen, a straight-chain or branched C₁₋₈-alkyl group or a straight-chain or branched C₁₋₈-alkoxy group. In accordance with the present invention, it is of course possible to use one dibenzoylmethane derivative or a plurality of dibenzoylmethane derivatives. Of the dibenzoylmethane derivatives to which the present invention more specifically relates, mention may be made, in particular, of:
-2-methyldibenzoylmethane,
-4-methyidibenzoylmethane,
-4-isopropyldibenzoylmethane,
-4-tert-butyidibenzoylmethane,
-2,4-dimethyldibenzoylmethane,
-2,5-dimethyidibenzoylmethane,
-4,4′-diisopropyidibenzoylmethane,
-4,4′-methoxy-tert-butyldibenzoylmethane,
-2-methyl-5-isopropyl-4′-methoxydibenzoylmethane,
-2-methyl-5-tert-butyl4′-methoxydibenzoylmethane,
-2,4-dimethyl-4′-methoxydibenzoylmethane
and 2,6-dimethyl-4-tert-butyl-4′-methoxydibenzoylmethane, this list being non-restrictive. Of the above-mentioned dibenzoylmethane derivatives, particular preference is given in accordance with the invention to 4,4′-methoxy-tert-butyldibenzoylmethane and especially 4,4′-methoxy-tert-butyldibenzoylmethane, which is commercially available under the trade name Eusolex® 9020 from Merck KGaA, where this filter conforms to the following structural formula:

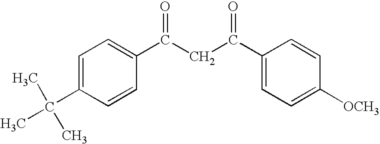

A further dibenzoylmethane derivative which is preferred in accordance with the invention is 4-isopropyidibenzoylmethane.

Further preferred compositions having light-protection properties comprise at least one benzophenone or benzophenone derivatives, such as, particularly preferably, 2-hydroxy4-methoxybenzophenone (for example Eusolex® 4360) or 2-hydroxy4-methoxybenzophenone-5-sulfonic acid and the sodium salt thereof (for example Uvinul® MS40).

The dibenzoylmethane derivative(s) or the benzophenone derivative(s) may be present in the compositions according to the invention in proportions which are generally in the range from 0.1 to 10% by weight and preferably in proportions which are in the range from 0.3 to 5% by weight, where these proportions are based on the total weight of the composition.

It may furthermore be preferred in accordance with the invention for the compositions to comprise further inorganic UV filters. Preference is given here both to those from the group of the titanium dioxides, such as, for example, coated titanium dioxide (for example Eusolex® T-2000, Eusolex® T-AQUA), zinc oxides (for example Sachtotec®), iron oxides or also cerium oxides. These inorganic UV filters are generally incorporated into cosmetic compositions in an amount of 0.5 to 20 percent by weight, preferably 2-10%. In particular, it may be preferred here for particles according to the invention to be incorporated into one phase in emulsions and for a further inorganic UV filter to be incorporated into the other phase.

In accordance with the invention, the above-mentioned UV filters can also be provided with a surface treatment which augments the hydrophilic or hydrophobic properties. Thus, the application of corresponding compounds enables hydrophobicisation or hydrophilisation of the particle surfaces to be achieved. Suitable for hydrophobic modification is, for example, coating with organic acids, such as, for example, stearic acid or lauric acid, with LCST polymers, organic fluoroalcohol phosphates or silicone or silane coating.

The silicones are, as is known, organosilicon polymers or oligomers having a straight-chain or cyclic, branched or crosslinked structure with various molecular weights which are obtained by polymerisation and/or polycondensation with suitably functionalised silanes and are essentially formed from recurring main units in which the silicon atoms are linked to one another via oxygen atoms (siloxane bond), where optionally substituted hydrocarbon groups are bonded directly to the silicon atoms via a carbon atom. The commonest hydrocarbon groups are alkyl groups and in particular methyl groups, fluoroalkyl groups, aryl groups and in particular phenyl groups, and alkenyl groups and in particular vinyl groups. Further types of group which can be bonded to the siloxane chain either directly or via a hydrocarbon group are, in particular, hydrogen, the halogens and in particular chlorine, bromine or fluorine, the thiols, alkoxy groups, polyoxyalkylene groups (or polyethers) and in particular polyoxyethylene and/or polyoxypropylene, hydroxyl groups or hydroxyalkyl groups, optionally substituted amino groups, amide groups, acyloxy groups or acyloxyalkyl groups, hydroxyalkylamino groups or aminoalkyl groups, quaternary ammonium groups, amphoteric groups or betaine groups, anionic groups, such as carboxylates, thioglycolates, sulfosuccinates, thiosulfates, phosphates and sulfates, where this list is of course in no way limiting (so-called ‘organo-modified’ silicones).

For the purposes of the present invention, the term ‘silicones’ is also intended to encompass and cover the silanes and in particular the alkylsilanes required for their preparation.

The silicones which are suitable present invention, which can be used for sheathing the UV protectants, are preferably selected from the alkylsilanes, the polydialkylsiloxanes and the polyalkylhydrogenosiloxanes. The silicones are more preferably selected from octyltrimethylsilane, the polydimethylsiloxanes and the polymethylhydrogenosiloxanes.

The UV protectants can be present in the compositions according to the invention in proportions which are generally in the range from 0.1 to 50% by weight and preferably in proportions which are in the range from 0.5 to 20% by weight, where these proportions are based on the total weight of the composition.

In a further, likewise preferred embodiment of the present invention, the composition according to the invention comprises at least one self-tanning agent.

Advantageous self-tanning agents which can be employed are, inter alia:

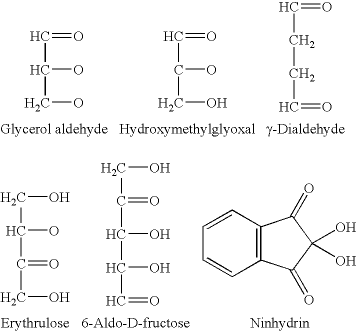

Mention should also be made of 5-hydroxy-1,4-naphthoquinone auglone), which is extracted from the shells of fresh walnuts

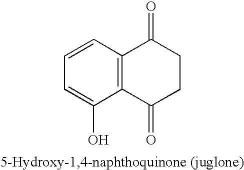

and 2-hydroxy-1,4-naphthoquinone (lawsone), which occurs in henna leaves.

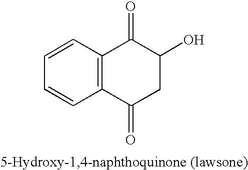

Very particular preference is given to 1,3-dihydroxyacetone (DHA), a trifunctional sugar which occurs in the human body, and derivatives thereof.

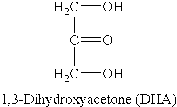

The present invention furthermore relates to the use of particles according to the invention in the stabilisation of self-tanning agents, in particular dihydroxyacetone or dihydroxyacetone derivatives.

Furthermore, the compositions according to the invention may also comprise dyes and coloured pigments. The dyes and coloured pigments can be selected from the corresponding positive list in the German Cosmetics Regulation or the EC list of cosmetic colorants. In most cases, they are identical with the dyes approved for foods. Advantageous coloured pigments are, for example, titanium dioxide, mica, iron oxides (for example Fe₂O₃, Fe₃O₄, FeO(OH)) and/or tin oxide. Advantageous dyes are, for example, carmine, Berlin Blue, Chromium Oxide Green, Ultramarine Blue and/or Manganese Violet. It is particularly advantageous to select the dyes and/or coloured pigments from the following list. The Colour Index numbers (CINs) are taken from the Rowe Colour Index, 3rd Edition, Society of Dyers and Colourists, Bradford, England, 1971.

| Chemical or other name | CIN | Colour |
|---|---|---|
| Pigment Green | 10006 | Green |
| Acid Green 1 | 10020 | Green |
| 2,4-Dinitrohydroxynaphthalene-7-sulfonic acid | 10316 | Yellow |
| Pigment Yellow 1 | 11680 | Yellow |
| Pigment Yellow 3 | 11710 | Yellow |
| Pigment Orange 1 | 11725 | Orange |
| 2,4-Dihydroxyazobenzene | 11920 | Orange |
| Solvent Red 3 | 12010 | Red |
| 1-(2′-chloro-4′-nitro-1′-phenylazo)-2-hydroxynaphthalene | 12085 | Red |
| Pigment Red 3 | 12120 | Red |
| Ceres Red; Sudan Red; Fat Red G | 12150 | Red |
| Pigment Red 112 | 12370 | Red |
| Pigment Red 7 | 12420 | Red |
| Pigment Brown 1 | 12480 | Brown |
| 4-(2′-Methoxy-5′-sulfodiethylamido-1′-phenylazo)-3- | 12490 | Red |
| hydroxy-5″-chloro-2″,4″-dimethoxy-2-naphthanilide | | |
| Disperse Yellow 16 | 12700 | Yellow |
| 1-(4-Sulfo-1-phenylazo)-4-aminobenzene-5-sulfonic acid | 13015 | Yellow |
| 2,4-Dihydroxyazobenzene-4′-sulfonic acid | 14270 | Orange |
| 2-(2,4-Dimethylphenylazo-5-sulfonyl)-1-hydroxy- | 14700 | Red |
| naphthalene-4-sulfonic acid | | |
| 2-(4-Sulfo-1-naphthylazo)-1-naphthol-4-sulfonic acid | 14720 | Red |
| 2-(6-Sulfo-2,4-xylylazo)-1-naphthol-5-sulfonic acid | 14815 | Red |
| 1-(4′-Sulfophenylazo)-2-hydroxynaphthalene | 15510 | Orange |
| 1-(2-Sulfonyl-4-chloro-5-carboxy-1-phenylazo)-2- | 15525 | Red |
| hydroxynaphthalene | | |
| 1-(3-Methylphenylazo-4-sulfonyl)-2-hydroxynaphthalene | 15580 | Red |
| 1-(4′,(8′)-Sulfonylnaphthylazo)-2-hydroxynaphthalene | 15620 | Red |
| 2-Hydroxy-1,2′-azonaphthalene-1′-sulfonic acid | 15630 | Red |
| 3-Hydroxy-4-phenylazo-2-naphthylcarboxylic acid | 15800 | Red |
| 1-(2-Sulfo-4-methyl-1-phenylazo)-2-naphthylcarboxylic | 15850 | Red |
| acid | | |
| 1-(2-Sulfo-4-methyl-5-chloro-1-phenylazo)-2-hydroxy- | 15865 | Red |
| naphthalene-3-carboxylic acid | | |
| 1-(2-Sulfo-1-naphthylazo)-2-hydroxynaphthalene-3- | 15880 | Red |
| carboxylic acid | | |
| 1-(3-Sulfo-1-phenylazo)-2-naphthol-6-sulfonic acid | 15980 | Orange |
| 1-(4-Sulfo-1-phenylazo)-2-naphthol-6-sulfonic acid | 15985 | Yellow |
| Allura Red | 16035 | Red |
| 1-(4-Sulfo-1-naphthylazo)-2-naphthol-3,6-disulfonic acid | 16185 | Red |
| Acid Orange 10 | 16230 | Orange |
| 1-(4-Sulfo-1-naphthylazo)-2-naphthol-6,8-disulfonic acid | 16255 | Red |
| 1-(4-Sulfo-1-naphthylazo)-2-naphthol-3,6,8-trisulfonic | 16290 | Red |
| acid | | |
| 8-Amino-2-phenylazo-1-naphthol-3,6-disulfonic acid | 17200 | Red |
| Acid Red 1 | 18050 | Red |
| Acid Red 155 | 18130 | Red |
| Acid Yellow 121 | 18690 | Yellow |
| Acid Red 180 | 18736 | Red |
| Acid Yellow 11 | 18820 | Yellow |
| Acid Yellow 17 | 18965 | Yellow |
| 4-(4-Sulfo-1-phenylazo)-1-(4-sulfophenyl)-5-hydroxy- | 19140 | Yellow |
| pyrazolone-3-carboxylic acid | | |
| Pigment Yellow 16 | 20040 | Yellow |
| 2,6-(4′-Sulfo-2″,4″-dimethyl)bisphenylazo)-1,3-dihydroxy- | 20170 | Orange |
| benzene | | |
| Acid Black 1 | 20470 | Black |
| Pigment Yellow 13 | 21100 | Yellow |
| Pigment Yellow 83 | 21108 | Yellow |
| Solvent Yellow | 21230 | Yellow |
| Acid Red 163 | 24790 | Red |
| Acid Red 73 | 27290 | Red |
| 2-[4′-(4″-Sulfo-1″-phenylazo)-7′-sulfo-1′-naphthylazo]-1- | 27755 | Black |
| hydroxy-7-aminonaphthalene-3,6-disulfonic acid | | |
| 4-[4″-Sulfo-1″-phenylazo)-7′-sulfo-1′-naphthylazo]-1- | 28440 | Black |
| hydroxy-8-acetylaminonaphthalene-3,5-disulfonic acid | | |
| Direct Orange 34, 39, 44, 46, 60 | 40215 | Orange |
| Food Yellow | 40800 | Orange |
| trans-β-Apo-8′-carotene aldehyde (C₃₀) | 40820 | Orange |
| trans-Apo-8′-carotinic acid (C₃₀) ethyl ester | 40850 | Orange |
| Canthaxanthine | 40850 | Orange |
| Acid Blue 1 | 42045 | Blue |
| 2,4-Disulfo-5-hydroxy-4′-4″- | 42051 | Blue |
| bis(diethylamino)triphenylcarbinol | | |
| 4-[(4-N-Ethyl-p-sulfobenzylamino)-phenyl-(4-hydroxy-2- | 42053 | Green |
| sulfophenyl)(methylene)-1-(N-ethyl-N-p-sulfobenzyl)-2,5- | | |
| cyclohexadienimine] | | |
| Acid Blue 7 | 42080 | Blue |
| (N-Ethyl-p-sulfobenzylamino)phenyl-(2-sulfophenyl)- | 42090 | Blue |
| methylene-(N-ethyl-N-p-sulfobenzyl)-Δ^{2,5}-cyclohexa- | | |
| dienimine | | |
| Acid Green 9 | 42100 | Green |
| Diethyldisulfobenzyldi-4-amino-2-chlorodi-2-methyl- | 42170 | Green |
| fuchsonimmonium | | |
| Basic Violet 14 | 42510 | Violet |
| Basic Violet 2 | 42520 | Violet |
| 2′-Methyl-4′-(N-ethyl-N-m-sulfobenzyl)amino-4″-(N-diethyl)amino- | 42735 | Blue |
| 2-methyl-N-ethyl-N-m-sulfobenzyl- | | |
| fuchsonimmonium | | |
| 4′-(N-Dimethyl)amino-4″-(N-phenyl)aminonaphtho-N-di- | 44045 | Blue |
| methylfuchsonimmonium | | |
| 2-Hydroxy-3,6-disulfo-4,4′-bisdimethylaminonaphtho- | 44090 | Green |
| fuchsonimmonium | | |
| Acid Red 52 | 45100 | Red |
| 3-(2′-Methylphenylamino)-6-(2′-methyl-4′-sulfophenyl- | 45190 | Violet |
| amino)-9-(2″-carboxyphenyl)xanthenium salt | | |
| Acid Red 50 | 45220 | Red |
| Phenyl-2-oxyfluorone-2-carboxylic acid | 45350 | Yellow |
| 4,5-Dibromofluorescein | 45370 | Orange |
| 2,4,5,7-Tetrabromofluorescein | 45380 | Red |
| Solvent Dye | 45396 | Orange |
| Acid Red 98 | 45405 | Red |
| 3′,4′,5′,6′-Tetrachloro-2,4,5,7-tetrabromofluorescein | 45410 | Red |
| 4,5-Diiodofluorescein | 45425 | Red |
| 2,4,5,7-Tetraiodofluorescein | 45430 | Red |
| Quinophthalone | 47000 | Yellow |
| Quinophthalonedisulfonic acid | 47005 | Yellow |
| Acid Violet 50 | 50325 | Violet |
| Acid Black 2 | 50420 | Black |
| Pigment Violet 23 | 51319 | Violet |
| 1,2-Dioxyanthraquinone, calcium-aluminium complex | 58000 | Red |
| 3-Oxypyrene-5,8,10-sulfonic acid | 59040 | Green |
| 1-Hydroxy-4-N-phenylaminoanthraquinone | 60724 | Violet |
| 1-Hydroxy-4-(4′-methylphenylamino)anthraquinone | 60725 | Violet |
| Acid Violet 23 | 60730 | Violet |
| 1,4-Di(4′-methylphenylamino)anthraquinone | 61565 | Green |
| 1,4-Bis(o-sulfo-p-toluidino)anthraquinone | 61570 | Green |
| Acid Blue 80 | 61585 | Blue |
| Acid Blue 62 | 62045 | Blue |
| N,N′-Dihydro-1,2,1′,2′-anthraquinonazine | 69800 | Blue |
| Vat Blue 6; Pigment Blue 64 | 69825 | Blue |
| Vat Orange 7 | 71105 | Orange |
| Indigo | 73000 | Blue |
| Indigodisulfonic acid | 73015 | Blue |
| 4,4′-Dimethyl-6,6′-dichlorothioindigo | 73360 | Red |
| 5,5′Dichloro-7,7′-dimethylthioindigo | 73385 | Violet |
| Quinacridone Violet 19 | 73900 | Violet |
| Pigment Red 122 | 73915 | Red |
| Pigment Blue 16 | 74100 | Blue |
| Phthalocyanine | 74160 | Blue |
| Direct Blue 86 | 74180 | Blue |
| chlorinated phthalocyanine | 74260 | Green |
| Natural Yellow 6, 19; Natural Red 1 | 75100 | Yellow |
| Bixin, Nor-Bixin | 75120 | Orange |
| Lycopene | 75125 | Yellow |
| trans-alpha-, -beta- or -gamma-Carotene | 75130 | Orange |
| Keto and/or hydroxyl derivatives of carotene | 75135 | Yellow |
| Guanine or pearlescent agent | 75170 | White |
| 1,7-Bis(4-hydroxy-3-methoxyphenyl)-1,6-heptadiene-3,5-dione | 75300 | Yellow |
| Complex salt (Na, Al, Ca) of carminic acid | 75470 | Red |
| chlorophyll a and b; copper compounds of chlorophylls | 75810 | Green |
| and chlorophyllines | | |
| Aluminium | 77000 | White |
| Aluminium hydroxide | 77002 | White |
| Water-containing aluminium silicates | 77004 | White |
| Ultramarine | 77007 | Blue |
| Pigment Red 101 and 102 | 77015 | Red |
| Barium sulfate | 77120 | White |
| Bismuth oxychloride and mixtures thereof with mica | 77163 | White |
| Calcium carbonate | 77220 | White |
| Calcium sulfate | 77231 | White |
| Carbon | 77266 | Black |
| Pigment Black 9 | 77267 | Black |
| Carbo medicinalis vegetabilis | 77268:1 | Black |
| Chromium oxide | 77288 | Green |
| Chromium oxide, water-containing | 77278 | Green |
| Pigment Blue 28, Pigment Green 14 | 77346 | Green |
| Pigment Metal 2 | 77400 | Brown |
| Gold | 77480 | Brown |
| Iron oxides and hydroxides | 77489 | Orange |
| Iron oxide | 77491 | Red |
| Iron oxide hydrate | 77492 | Yellow |
| Iron oxide | 77499 | Black |
| Mixtures of iron(II) and iron(III) hexacyanoferrate | 77510 | Blue |
| Pigment White 18 | 77713 | White |
| Manganese ammonium diphosphate | 77742 | Violet |
| Manganese phosphate; Mn₃(PO₄)₂•7H₂O | 77745 | Red |
| Silver | 77820 | White |
| Titanium dioxide and mixtures thereof with mica | 77891 | White |
| Zinc oxide | 77947 | White |
| 6,7-Dimethyl-9-(1′-D-ribityl)isoalloxazine, lactoflavin | | Yellow |
| Sugar dye | | Brown |
| Capsanthin, capsorubin | | Orange |
| Betanin | | Red |
| Benzopyrylium salts, anthocyans | | Red |
| Aluminium, zinc, magnesium and calcium stearate | | White |
| bromothymol Blue | | Blue |

It may furthermore be favourable to select, as dye, one or more substances from the following group:
2,4-dihydroxyazobenzene, 1-(2′-chloro4′-nitro-1′-phenylazo)-2-hydroxy-naphthalene, Ceres Red, 2-(4-sulfo-1-naphthylazo)-1-naphthol4-sulfonic acid, the calcium salt of 2-hydroxy-1,2′-azonaphthalene-1′-sulfonic acid, the calcium and barium salts of 1-(2-sulfo4-methyl-1-phenylazo)-2-naphthyl-carboxylic acid, the calcium salt of 1-(2-sulfo-1-naphthylazo)-2-hydroxy-naphthalene-3-carboxylic acid, the aluminium salt of 1-(4-sulfo-1-phenylazo)-2-naphthol-6-sulfonic acid, the aluminium salt of 1-(4-sulfo-1-naphthylazo)-2-naphthol-3,6-disulfonic acid, 1-(4-sulfo-1-naphthylazo)-2-naphthol-6,8-disulfonic acid, the aluminium salt of 4-(4-sulfo-1-phenylazo)-2-(4-sulfophenyl)-5-hydroxypyrazolone-3-carboxylic acid, the aluminium and zirconium salts of 4,5-dibromofluorescein, the aluminium and zirconium salts of 2,4,5,7-tetrabromofluorescein, 3′,4′,5′,6′-tetrachloro-2,4,5,7-tetrabromofluorescein and the aluminium salt thereof, the aluminium salt of 2,4,5,7-tetraiodofluorescein, the aluminium salt of quinophthalonedisulfonic acid, the aluminium salt of indigodisulfonic acid, red and black iron oxide (CIN: 77 491 (red) and 77 499 (black)), iron oxide hydrate (CIN: 77492), manganese ammonium diphosphate and titanium dioxide.

Also advantageous are oil-soluble natural dyes, such as, for example, paprika extract, β-carotene or cochineal.

Also advantageous for the purposes of the present invention are gel creams comprising effect pigments. Particular preference is given to the types of effect pigment listed below:

1. Natural effect pigments, such as, for example,
a) “pearl essence” (guanine/hypoxanthine mixed crystals from fish scales) and
b) “mother-of-pearl” (ground mussel shells)

2. Monocrystalline effect pigments, such as, for example, bismuth oxychloride (BiOCI)

3. Layered substrate pigments: for example mica/metal oxide

The basis for effect pigments is formed by, for example, pulverulent pigments or castor oil dispersions of bismuth oxychloride and/or titanium dioxide as well as bismuth oxychloride and/or titanium dioxide on mica. The lustre pigment listed under CIN 77163, for example, is particularly advantageous.

Also advantageous are, for example, the following effect pigment types based on mica/metal oxide:

| Group | Coating/layer thickness | Colour |
|---|---|---|
| Silver-white effect pigments | TiO₂: 40-60 nm | silver |
| Interference pigments | TiO₂: 60-80 nm | yellow |
| | TiO₂: 80-100 nm | red |
| | TiO₂: 100-140 nm | blue |
| | TiO₂: 120-160 nm | green |
| Coloured lustre pigments | Fe₂O₃ | bronze |
| | Fe₂O₃ | copper |
| | Fe₂O₃ | red |
| | Fe₂O₃ | red-violet |
| | Fe₂O₃ | red-green |
| | Fe₂O₃ | black |
| Combination pigments | TiO₂/Fe₂O₃ | gold shades |
| | TiO₂/Cr₂O₃ | green |
| | TiO₂/Berlin Blue | dark blue |

Particular preference is given to, for example, the pearlescent pigments available from Merck KGaA under the trade names Timiron®, Colorona® or Dichrona®.

The list of the said effect pigments is of course not intended to be limiting. Effect pigments which are advantageous for the purposes of the present invention can be obtained by numerous routes known per se. In addition, for example, other substrates apart from mica can also be coated with further metal oxides, such as, for example, silica and the like. For example, TiO₂- and Fe₂O₃-coated SiO₂ particles (“Ronasphere” grades), which are marketed by Merck KGaA and are particularly suitable for the optical reduction of fine wrinkles, are advantageous.

It may additionally be advantageous to completely omit a substrate such as mica. Particular preference is given to effect pigments prepared using SiO₂ or Al₂O₃. Such pigments, which may additionally also have goniochromatic effects, are available, for example, from Merck KGaA under the trade name Colorstream® or Xirallic®.

It may also be advantageous to employ Engelhard pigments based on calcium sodium borosilicate coated with titanium dioxide. These are available under the name Reflecks®. Due to their particle size of 40-80 μm, they have a glitter effect in addition to the colour.

Also particularly advantageous are effect pigments available from Flora Tech under the trade name Metasomes® Standard/Glitter in various colours (yellow, red, green, blue). The glitter particles here are in the form of mixtures with various auxiliaries and dyes (such as, for example, the dyes with the Colour Index (CI) numbers 19140, 77007, 77289, 77491).

The dyes and pigments can be in individual form or in the form of a mixture and mutually coated with one another, with different colour effects generally being caused by different coating thicknesses. The total amount of dyes and colouring pigments is advantageously selected from the range from, for example, 0.1% by weight to 30% by weight, preferably from 0.5 to 15% by weight, in particular from 1.0 to 10% by weight, in each case based on the total weight of the compositions.

The compositions according to the invention may of course comprise one or more additional hydrophilic or lipophilic sunscreen filters which are effective in the UV-A region and/or UV-B region and/or IR and/or VIS region (absorbers). These additional filters can be selected, in particular, from cinnamic acid derivatives, salicylic acid derivatives, camphor derivatives, triazine derivatives, β,β-diphenyl acrylate derivatives, p-aminobenzoic acid derivatives and polymeric filters and silicone filters, which are described in the application WO 93/04665. Further examples of organic filters are indicated in patent application EP-A 0 487 404.

In principle, all UV filters are suitable for combination with the particles according to the invention. Particular preference is given to UV filters whose physiological acceptability has already been demonstrated. Both for UVA and UVB filters, there are many proven substances which are known from the specialist literature, for example benzylidenecamphor derivatives, such as 3-(4′-methylbenzylidene)-dl-camphor (for example Eusolex® 6300), 3-benzylidenecamphor (for example Mexoryl® SD), polymers of N-{(2 and 4)-[(2-oxoborn-3-ylidene)methyl]benzyl}acrylamide (for example Mexoryl® SW), N,N,N-trimethyl4-(2-oxoborn-3-ylidenemethyl)anilinium methylsulfate (for example Mexoryl® SK) or (2-oxoborn-3-ylidene)toluene-4-sulfonic acid (for example Mexoryl® SL), methoxycinnamic acid esters, such as octyl methoxycinnamate (for example Eusolex® 2292), isopentyl 4-methoxycinnamate, for example as a mixture of the isomers (for example Neo Heliopan® E 1000), salicylate derivatives, such as 2-ethylhexyl salicylate (for example Eusolex® OS), 4-isopropylbenzyl salicylate (for example Megasol®) or 3,3,5-trimethylcyclohexyl salicylate (for example Eusolex® HMS), 4-aminobenzoic acid and derivatives, such as 4-aminobenzoic acid, 2-ethylhexyl 4-(dimethylamino)benzoate (for example Eusolex® 6007), ethoxylated ethyl 4-aminobenzoate (for example Uvinul® P25), phenylbenzimidazolesulfonic acids, such as 2-phenylbenzimidazole-5-sulfonic acid and the potassium, sodium and triethanolamine salts thereof (for example Eusolex® 232), 2,2-(1,4-phenylene)bisbenzimidazole4,6-disulfonic acid and salts thereof (for example Neoheliopan® AP) or 2,2-(1,4-phenylene)bis-benzimidazole-6-sulfonic acid; and further substances, such as
-2-ethylhexyl 2-cyano-3,3-diphenylacrylate (for example Eusolex® OCR),
-3,3′-(1,4-phenylenedimethylene)bis-7,7-dimethyl-2-oxobicyclo[2.2.1]hept-1-ylmethanesulfonic acid and salts thereof (for example Mexorylo SX) and
2,4,6-trianilino-(p-carbo-2′-ethylhexyl-1′-oxy)-1,3,5-triazine (for example Uvinul® T 150)
-hexyl 2-(4-diethylamino-2-hydroxybenzoyl)benzoate (for example Uvinul® UVA Plus, BASF).

The compounds mentioned in the list should only be regarded as examples. It is of course also possible to use other UV filters. In particular, organic particulate UV filters, as described, for example, in patent application WO 99/66896, may also advantageously be combined with the powders according to the invention.

These organic UV filters are generally incorporated into cosmetic formulations in an amount of 0.5 to 20 percent by weight, preferably 1-10% by weight.

Further suitable organic UV filters are, for example,
2-(2H-benzotriazol-2-yl)-4-methyl-6-(2-methyl-3-(1,3,3,3-tetramethyl-1-(trimethylsilyloxy)disiloxanyl)propyl)phenol (for example Silatrizole®),
2-ethylhexyl 4,4′-[(6-[4-((1,1-dimethylethyl)aminocarbonyl)phenylamino]-1,3,5-triazine-2,4iyl)diimino]bis(benzoate) (for example Uvasorb® HEB),
α-(trimethylsilyl)-ω-[trimethylsilyl)oxy]poly[oxy(dimethyl [and approx. 6% of methyl[2-[p-[2,2-bis(ethoxycarbonyl]vinyl]phenoxy]-1-methyleneethyl] and approx. 1.5% of methyl[3-[p-[2,2-bis(ethoxycarbonyl)vinyl)phenoxy)-propenyl) and 0.1 to 0.4% of (methylhydrogen]silylene]] (n≈60) (CAS No. 207 574-74-1)
2,2′-methylenebis(6-(2H-benzotriazol-2-yl)4-(1,1,3,3-tetramethylbutyl)-phenol) (CAS No. 103 59745-1)
2,2′-(1,4-phenylene)bis(1H-benzimidazole4,6-disulfonic acid, mono-sodium salt) (CAS No. 180 898-37-7) and
2,4-bis{[4-(2-ethylhexyloxyy2-hydroxy]phenyl}-6-(4-methoxyphenyl)-1,3,5-triazine (CAS No. 103 59745-, 187 393-00-6).
2-ethylhexyl 4,4′-[(6-[4-((1,1-dimethylethyl)aminocarbonyl)phenylamino]-1,3,5-triazine-2,4-diyl)diimino]bis(benzoate) (for example Uvasorb® HEB),

Preferred compounds having UV-filtering properties are 3-(4′-methylbenzylidene)-dl-camphor, 1-(4-tert-butylphenyl)-3-(4-methoxyphenyl)propane-1,3-dione, 4-isopropyldibenzoylmethane, 2-hydroxy-4-methoxybenzophenone, octyl methoxycinnamate, 3,3,5-trimethylcyclohexyl salicylate, 2-ethylhexyl 4-(dimethylamino)benzoate, 2-ethylhexyl 2-cyano-3,3-diphenylacrylate, 2-phenylbenzimidazole-5-sulfonic acid and the potassium, sodium and triethanolamine salts thereof.

Preferred compositions may also comprise compounds of the formula I

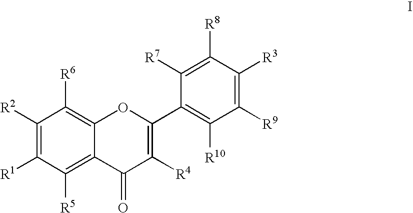

where R₁ and R² are selected from
H
and OR¹¹, where OR¹¹, independently of one another, stands for
   OH
   straight-chain or branched C₁- to C₂₀-alkoxy groups,
   straight-chain or branched C₃- to C₂₀-alkenyloxy groups,
   straight-chain or branched C₁- to C₂₀-hydroxyalkoxy groups, where the hydroxyl group(s) may be bonded to primary or secondary carbon atoms of the chain and furthermore the alkyl chain may also be interrupted by oxygen, and/or
   C₃- to C₁₀-cycloalkoxy groups and/or C₃- to C₁₂-cycloalkenyloxy groups, where the rings may each also be bridged by —(CH₂)ₙ- groups, where n=1 to 3, and/or mono- and/or oligoglycosyl radicals,

with the proviso that at least one radical from R¹ and R² stands for OR¹¹,

and R³ stands for a radical OR¹¹ and

R⁴ to R⁷ and R¹⁰ may be identical or different and, independently of one another, stand for
H
straight-chain or branched C₁- to C₂₀-alkyl groups,
straight-chain or branched C₃- to C₂₀-alkenyl groups,
straight-chain or branched C₁- to C₂₀-hydroxyalkyl groups, where the hydroxyl group may be bonded to a primary or secondary carbon atom of the chain and furthermore the alkyl chain may also be interrupted by oxygen, and/or
C₃- to C₁₀-cycloalkyl groups and/or C₃- to C₁₂-cycloalkenyl groups, where the rings may each also be bridged by —(CH₂)n- groups, where n=1 to 3, and

R⁸ and R⁹ may be identical or different and, independently of one another, stand for
H
OR¹¹
straight-chain or branched C₁- to C₂₀-alkyl groups,
straight-chain or branched C₃- to C₂₀-alkenyl groups,
straight-chain or branched C₁- to C₂₀-hydroxyalkyl groups, where the hydroxyl group may be bonded to a primary or secondary carbon atom of the chain and furthermore the alkyl chain may also be interrupted by oxygen, and/or
C₃- to C₁₀-cycloalkyl groups and/or C₃- to C₁₂-cycloalkenyl groups, where the rings may each also be bridged by —(CH₂)ₙ- groups, wheren=1 to 3.

Advantages of the compositions according to the invention are, in particular, the UV light-filtering action and the good toleration by the skin. In addition, the compounds of the formula I described here are colourless or only weakly coloured and thus, in contrast to many known naturally occurring flavonoids, do not result in discoloration of the compositions.

The flavonoids of the formula I to be employed in accordance with the invention include broad-band UV filters, other likewise preferred compounds of the formula I exhibit an absorption maximum in the boundary region between UV-B and UV-A radiation. As UV-A-II filters, they therefore advantageously supplement the absorption spectrum of commercially available UV-B and UV-A-I filters. Preferred compositions according to the invention having light-protection properties comprise at least one compound of the formula I, where R³ stands for
OH or
straight-chain or branched C₁- to C₂₀-alkoxy groups, preferably methoxy, ethoxy or ethylhexyloxy, or
mono- and/or oligoglycosyl radicals, preferably glucosyl radicals, and
R¹ and/or R² preferably stand for

OH or

straight-chain or branched C₁- to C₂₀-alkoxy groups, preferably methoxy, ethoxy or ethylhexyloxy, or

mono- and/or oligoglycosyl radicals, preferably glucosyl radicals.

These preferred compounds are distinguished by particularly intense UV absorption.

In addition, preferred compounds of this type have advantages on incorporation into the compositions:
mono- and/or oligoglycosyl radicals improve the water solubility of the compounds to be employed in accordance with the invention;
straight-chain or branched C₁- to C₂₀-alkoxy groups, in particular the long-chain alkoxy functions, such as ethylhexyloxy groups, increase the oil solubility of the compounds;

i.e. the hydrophilicity or lipophilicity of the compounds of the formula I can be controlled via a suitable choice of the substituents. Preferred mono- or oligosaccharide radicals here are hexosyl radicals, in particular ramnosyl radicals and glucosyl radicals. However, other hexosyl radicals, for example allosyl, altrosyl, galactosyl, gulosyl, idosyl, mannosyl and talosyl, may also, if desired, advantageously be used. It may also be advantageous to use pentosyl radicals. The glycosyl radicals can be bonded to the parent structure α- or β-glycosidically. A preferred disaccharide is, for example, 6-O-(6-deoxy-α-L-mannopyranosyl)-β-D-glucopyranoside.

It has been found that the intensity of the UV absorption is particularly high if R³ stands for straight-chain or branched C₁- to C₂₀-alkoxy groups, preferably methoxy, ethoxy or ethylhexyloxy, and R⁸ and R⁹ are identical and stand for H or straight-chain or branched C₁- to C₂₀-alkoxy groups, preferably methoxy, ethoxy or ethylhexyloxy.

Particular preference is therefore given in accordance with the invention to compositions having light-protection properties comprising at least one compound of the formula I which is characterised in that R³ stands for straight-chain or branched C₁- to C₂₀-alkoxy groups, preferably methoxy, ethoxy or ethylhexyloxy, and R⁸ and R⁹ are identical and stand for H or straight-chain or branched C₁- to C₂₀-alkoxy groups, preferably methoxy, ethoxy or ethylhexyloxy. It is particularly preferred here if R⁸ and R⁹ stand for H.

The compounds of the formula I are typically employed in accordance with the invention in amounts of 0.01 to 20% by weight, preferably in amounts of 0.5% by weight to 10% by weight and particularly preferably in amounts of 1 to 8% by weight. The person skilled in the art is presented with absolutely no difficulties at all in correspondingly selecting the amounts depending on the intended light protection factor of the composition.

Combination of particles with further UV filters in the powders according to the invention enables the protective action against harmful effects of UV radiation to be optimised.

All said UV filters, including the compounds of the formula I, can likewise also be employed in encapsulated form. In particular, it is advantageous to employ organic UV filters in encapsulated form. Specifically, the encapsulation gives rise to the following advantages:
The hydrophilicity of the capsule wall can be set independently of the solubility of the UV filter. Thus, for example, it is also possible to incorporate hydrophobic UV filters into purely aqueous compositions. In addition, the oily impression on application of the composition comprising hydrophobic UV filters, which is frequently regarded as unpleasant, is suppressed.
Certain UV filters, in particular dibenzoylmethane derivatives, exhibit only reduced photostability in cosmetic compositions. Encapsulation of these filters or compounds which impair the photostability of these filters, such as, for example, cinnamic acid derivatives, enables the photostability of the entire composition to be increased.
Skin penetration by organic UV filters and the associated potential for irritation on direct application to the human skin are repeatedly discussed in the literature. The encapsulation of the corresponding substances which is proposed here suppresses this effect.
In general, encapsulation of individual UV filters or other ingredients enables preparation problems caused by the interaction of individual composition constituents with one another, such as crystallisation processes, precipitation and agglomerate formation, to be avoided since the interaction is suppressed.

It may therefore be preferred in accordance with the invention for one or more of the above-mentioned UV filters to be in encapsulated form. It is advantageous here for the capsules to be so small that they cannot be viewed with the naked eye. In order to achieve the abovementioned effects, it is furthermore necessary for the capsules to be sufficiently stable and the encapsulated active ingredient (UV filter) only to be released to the environment to a small extent, or not at all.

Suitable capsules can have walls of inorganic or organic polymers. For example, U.S. Pat. No. 6,242,099 B1 describes the production of suitable capsules with walls of chitin, chitin derivatives or polyhydroxylated polyamines. Capsules which can particularly preferably be employed in accordance with the invention have walls which can be obtained by a so-gel process, as described in the applications EP 1 382 328, WO 00/09652, WO 00/72806 and WO 00/71084. Preference is again given here to capsules whose walls are built up from silica gel (silica; undefined silicon oxide hydroxide) or silicon dioxide. The production of corresponding capsules is known to the person skilled in the art, for example from the cited patent applications, whose contents expressly also belong to the subject-matter of the present application. In addition, these capsules may also be after-treated, i.e. the surface of the particles is hydrophobicised or hydrophilised. Examples of after-treatments of this type are already known.

If the compositions according to the invention comprise compounds of the formula I containing free hydroxyl groups, they additionally, besides the properties described, exhibit an action as antioxidant and/or free-radical scavenger. Preference is therefore also given to compositions having light-protection properties comprising at least one compound of the formula I which is characterised in that at least one of the radicals R¹ to R³ stands for OH, preferably with at least one of the radicals R¹ or R² standing for OH.

In order that the compounds of the formula I are able to develop their positive action as free-radical scavengers particularly well on the skin, it may be preferred to allow the compounds of the formula I to penetrate into deeper skin layers. Several possibilities are available for this purpose. Firstly, the compounds of the formula I can have an adequate lipophilicity in order to be able to penetrate through the outer skin layer into epidermal layers. As a further possibility, corresponding transport agents, for example liposomes, which enable transport of the compounds of the formula I through the outer skin layers may also be provided in the composition. Finally, systemic transport of the compounds of the formula I is also conceivable. The composition is then designed, for example, in such a way that it is suitable for oral administration.

In general, the substances of the formula I act as free-radical scavengers. Free radicals of this type are not generated only by sunlight, but instead are formed under various conditions. Examples are anoxia, which blocks the flow of electrons upstream of the cytochrome oxidases and causes the formation of superoxide free-radical anions; inflammation associated, inter alia, with the formation of superoxide anions by the membrane NADPH oxidase of the leucocytes, but also associated with the formation (through disproportionation in the presence of iron(II) ions) of the hydroxyl free radicals and other reactive species which are normally involved in the phenomenon of phagocytosis; and lipid autoxidation, which is generally initiated by a hydroxyl free radical and produces lipidic alkoxy free radicals and hydroperoxides.

It is assumed that preferred compounds of the formula I also act as enzyme inhibitors. They are thought to inhibit histidine decarboxylase, protein kinases, elastase, aldose reductase and hyaluronidase, and therefore enable the intactness of the basic substance of vascular sheaths to be maintained. Furthermore, they are thought to inhibit catechol O-methyl transferase non-specifically, causing the amount of available catecholamines and thus the vascular strength to be increased. Furthermore, they inhibit AMP phosphodiesterase, giving the substances potential for inhibiting thrombocyte aggregation.

Owing to these properties, the compositions according to the invention are, in general, suitable for immune protection and for the protection of DNA and RNA. In particular, the compositions are suitable for the protection of DNA and RNA against oxidative attack, against free radicals and against damage due to radiation, in particular UV radiation. A further advantage of the compositions according to the invention is cell protection, in particular protection of Langerhans cells against damage due to the influences mentioned above. The present invention also expressly relates to all these uses and to the use of the compounds of the formula I for the preparation of compositions which can be employed correspondingly.

In particular, preferred compositions according to the invention are also suitable for the treatment of skin diseases associated with a defect in keratinisation which affects differentiation and cell proliferation, in particular for the treatment of acne vulgaris, acne comedonica, polymorphic acne, acne rosaceae, nodular acne, acne conglobata, age-induced acne, acne which arises as a side effect, such as acne solaris, medicament-induced acne or acne professionalis, for the treatment of other defects in keratinisation, in particular ichthyosis, ichthyosiform states, Darier's disease, keratosis palmoplantaris, leukoplakia, leukoplakiform states, herpes of the skin and mucous membrane (buccal) (lichen), for the treatment of other skin diseases associated with a defect in keratinisation and which have an inflammatory and/or immunoallergic component and in particular all forms of psoriasis which affect the skin, mucous membranes and fingers and toenails, and psoriatic rheumatism and skin atopy, such as eczema or respiratory atopy, or hypertrophy of the gums, it furthermore being possible for the compounds to be used for some inflammation which is not associated with a defect in keratinisation, for the treatment of all benign or malignant excrescence of the dermis or epidermis, which may be of viral origin, such as verruca vulgaris, verruca plana, epidermodysplasia verruciformis, oral papillomatosis, papillomatosis florida, and excrescence which may be caused by UV radiation, in particular epithelioma baso-cellulare and epithelioma spinocellulare, for the treatment of other skin diseases, such as dermatitis bullosa and diseases affecting the collagen, for the treatment of certain eye diseases, in particular corneal diseases, for overcoming or combating light-induced skin ageing associated with ageing, for reducing pigmentation and keratosis actinica and for the treatment of all diseases associated with normal ageing or light-induced ageing, for the prevention or healing of wounds/scars of atrophy of the epidermis and/or dermis caused by locally or systemically applied corticosteroids and all other types of skin atrophy, for the prevention or treatment of defects in wound healing, for the prevention or elimination of stretch marks caused by pregnancy or for the promotion of wound healing, for combating defects in sebum production, such as hyperseborrhoea in acne or simple seborrhoea, for combating or preventing cancer-like states or pre-carcinogenic states, in particular promyelocytic leukaemia, for the treatment of inflammatory diseases, such as arthritis, for the treatment of all virus-induced diseases of the skin or other areas of the body, for the prevention or treatment of alopecia, for the treatment of skin diseases or diseases of other areas of the body with an immunological component, for the treatment of cardiovascular diseases, such as arteriosclerosis or hypertension, and of non-insulin-dependent diabetes, and for the treatment of skin problems caused by UV radiation.

The protective action of compositions according to the invention against oxidative stress or against the effect of free radicals can thus be further improved if the compositions comprise one or more antioxidants, where the person skilled in the art is presented with absolutely no difficulties at all in selecting suitable antioxidants which act quickly or in a delayed manner.

In a preferred embodiment of the present invention, the composition is therefore a composition for the protection of body cells against oxidative stress, in particular for reducing skin ageing, characterised in that it comprises one or more antioxidants in addition to the one or more compounds of the formula I.

There are many proven substances known from the specialist literature which can be used as antioxidants, for example amino acids (for example glycine, histidine, tyrosine, tryptophan) and derivatives thereof, imidazoles (for example urocanic acid) and derivatives thereof, peptides, such as D,L-carnosine, D-carnosine, L-carnosine and derivatives thereof (for example anserine), carotinoids, carotenes (for example α-carotene, β-carotene, lycopene) and derivatives thereof, chlorogenic acid and derivatives thereof, lipoic acid and derivatives thereof (for example dihydrolipoic acid), aurothioglucose, propylthiouracil and other thiols (for example thioredoxin, glutathione, cysteine, cystine, cystamine and the glycosyl, N-acetyl, methyl, ethyl, propyl, amyl, butyl and lauryl, palmitoyl, oleyl, y-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (for example buthionine sulfoximines, homocysteine sulfoximine, buthionine sulfones, penta-, hexa- and heptathionine sulfoximine) in very low tolerated doses (for example pmol to μmol/kg), and also (metal) chelating agents (for example α-hydroxy fatty acids, palmitic acid, phytic acid, lactoferrin), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof, vitamin C and derivatives (for example ascorbyl palmitate, magnesium ascorbyl phosphate, ascorbyl acetate), tocopherols and derivatives (for example vitamin E acetate), vitamin A and derivatives (for example vitamin A palmitate), and coniferyl benzoate of benzoin resin, rutinic acid and derivatives thereof, α-glycosyl rutin, ferulic acid, furfurylideneglucitol, carnosine, butylhydroxytoluene, butylhydroxyanisole, nordihydroguaiaretic acid, trihydroxybutyrophenone, quercetin, uric acid and derivatives thereof, mannose and derivatives thereof, zinc and derivatives thereof (for example ZnO, ZnSO₄), selenium and derivatives thereof (for example selenomethionine), stilbenes and derivatives thereof (for example stilbene oxide, trans-stilbene oxide).

Mixtures of antioxidants are likewise suitable for use in the cosmetic compositions according to the invention. Known and commercial mixtures are, for example, mixtures comprising, as active ingredients, lecithin, L-(+)-ascorbyl palmitate and citric acid (for example Oxynex® AP), natural tocopherols, L-(+)-ascorbyl palmitate, L-(+)-ascorbic acid and citric acid (for example Oxynex® K LIQUID), tocopherol extracts from natural sources, L-(+)-ascorbyl palmitate, L-(+)-ascorbic acid and citric acid (for example Oxynex® L LIQUID), DL-α-tocopherol, L-(+)-ascorbyl palmitate, citric acid and lecithin (for example Oxynex® LM) or butylhydroxytoluene (BHT), L-(+)-ascorbyl palmitate and citric acid (for example Oxynex® 2004). Antioxidants of this type are usually employed in such compositions with compounds of the formula I in ratios in the range from 1000:1 to 1:1000, preferably in amounts of 100:1 to 1:100.

The compositions according to the invention may comprise vitamins as further ingredients. The cosmetic compositions according to the invention preferably comprise vitamins and vitamin derivatives selected from vitamin A, vitamin A propionate, vitamin A palmitate, vitamin A acetate, retinol, vitamin B, thiamine chloride hydrochloride (vitamin B₁), riboflavin (vitamin B₂), nicotinamide, vitamin C (ascorbic acid), vitamin D, ergocalciferol (vitamin D₂), vitamin E, DL-α-tocopherol, tocopherol E acetate, tocopherol hydrogensuccinate, vitamin K₁, esculin (vitamin P active ingredient), thiamine (vitamin B₁), nicotinic acid (niacin), pyridoxine, pyridoxal, pyridoxamine (vitamin B₆), pantothenic acid, biotin, folic acid and cobalamine (vitamin B₁₂), particularly preferably vitamin A palmitate, vitamin C and derivatives thereof, DL-α-tocopherol, tocopherol E acetate, nicotinic acid, pantothenic acid and biotin. Vitamins are usually employed here with compounds of the formula I in ratios in the range from 1000:1 to 1:1000, preferably in amounts of 100:1 to 1:100.

Of the phenols having an antioxidative action, the polyphenols, some of which are naturally occurring, are of particular interest for applications in the pharmaceutical, cosmetic or nutrition sector. For example, the flavonoids or bioflavonoids, which are principally known as plant dyes, frequently have an antioxidant potential. K. Lemanska, H. Szymusiak, B. Tyrakowska, R. Zielinski, I.M.C.M. Rietjens; Current Topics in Biophysics 2000, 24(2), 101-108, are concerned with effects of the substitution pattern of mono- and dihydroxyflavones. It is observed therein that dihydroxyflavones containing an OH group adjacent to the keto function or OH groups in the 3′,4′- or 6,7- or 7,8-position have antioxidative properties, while other mono- and dihydroxyflavones in some cases do not have antioxidative properties.

Quercetin (cyanidanol, cyanidenolon 1522, meletin, sophoretin, ericin, 3,3′,4′,5,7-pentahydroxyflavone) is frequently mentioned as a particularly effective antioxidant (for example C. A. Rice-Evans, N. J. Miller, G. Paganga, Trends in Plant Science 1997, 2(4), 152-159). K. Lemanska, H. Szymusiak, B. Tyrakowska, R. Zielinski, A.E.M.F. Soffers, I.M.C.M. Rietjens; Free Radical Biology&Medicine 2001, 31(7), 869-881, are investigating the pH dependence of the antioxidant action of hydroxyflavones. Quercetin exhibits the greatest activity amongst the structures investigated over the entire pH range.

Suitable antioxidants are furthermore compounds of the formula II

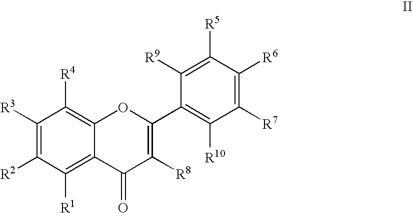

where R¹ to R¹⁰ may be identical or different and are selected from
H
OR¹¹
straight-chain or branched C₁- to C₂₀-alkyl groups,
straight-chain or branched C₃- to C₂₀-alkenyl groups,
straight-chain or branched C₁- to C₂₀-hydroxyalkyl groups, where the hydroxyl group may be bonded to a primary or secondary carbon atom of the chain and furthermore the alkyl chain may also be interrupted by oxygen, and/or
C₃- to C₁₀-cycloalkyl groups and/or C₃- to C₁₂-cycloalkenyl groups, where the rings may each also be bridged by —(CH₂)ₙ- groups, where n=1 to 3,
where all OR¹¹, independently of one another, stand for
OH
straight-chain or branched C₁- to C₂₀-alkoxy groups,
straight-chain or branched C₃- to C₂₀-alkenyloxy groups,
straight-chain or branched C₁- to C₂₀-hydroxyalkoxy groups, where the hydroxyl group(s) may be bonded to primary or secondary carbon atoms of the chain and furthermore the alkyl chain may also be interrupted by oxygen, and/or
C₃- to C₁₀-cycloalkoxy groups and/or C₃- to C₁₂-cycloalkenyloxy groups, where the rings may each also be bridged by —(CH₂)ₙ- groups, where n=1 to 3, and/or
mono- and/or oligoglycosyl radicals,

with the proviso that at least 4 radicals from R¹ to R⁷ stand for OH and that at least 2 pairs of adjacent —OH groups are present in the molecule,
or R², R⁵ and R⁶ stand for OH and the radicals R¹, R³, R⁴ and R⁷⁻¹⁰ stand for H,

as described in the earlier German patent application DE 10244282.7.

Advantages of the compositions according to the invention comprising at least one antioxidant, besides the above-mentioned advantages, are, in particular, the antioxidant action and the good tolerance by the skin. In addition, preferred compounds of those described here are colourless or only weakly coloured and thus do not result in discoloration of the compositions, or only do so to a small extent. Particularly advantageous is the particular action profile of the compounds of the formula II, which is evident in the DPPH assay from a high capacity for scavenging free radicals (EC₅₀), a delayed action (T_{EC50}>120 min) and thus morate to high anti-free-radical efficiency (AE). In addition, the compounds of the formula II combine in the molecule antioxidative properties with UV absorption in the UV-A and/or -B region. Preference is therefore also given to compositions comprising at least one compound of the formula II which is characterised in that at least two adjacent radicals of the radicals R¹ to R⁴ stand for OH and at least two adjacent radicals of the radicals R⁵ to R⁷ stand for OH. Particularly preferred compositions comprise at least one compound of the formula II which is characterised in that at least three adjacent radicals of the radicals R¹ to R⁴ stand for OH, where the radicals R¹ to R³ preferably stand for OH.

In accordance with the invention, flavone derivatives are taken to mean flavonoids and coumaranones. In accordance with the invention, flavonoids are taken to mean the glycosides of flavonones, flavones, 3-hydroxyflavones (=flavonols), aurones, isoflavones and rotenoids [Rompp Chemie Lexikon [Römpp's Lexicon of Chemistry], Volume 9, 1993]. For the purposes of the present invention, however, this term is also taken to mean the aglycones, i.e. the sugar-free constituents, and the derivatives of the flavonoids and aglycones. For the purposes of the present invention, the term flavonoid is furthermore also taken to mean anthocyanidine (cyanidine). For the purposes of the present invention, the term coumaranones is also taken to mean derivatives thereof.

Preferred flavonoids are derived from flavonones, flavones, 3-hydroxyflavones, aurones and isoflavones, in particular from flavonones, flavones, 3-hydroxyflavones and aurones.

The flavonoids are preferably selected from the following compounds: 4,6,3′,4′-tetrahydroxyaurone, quercetin, rutin, isoquercetin, eriodictyol, taxifolin, luteolin, trishydroxyethylquercetin (troxequercetin), trishydroxyethylrutin (troxerutin), trishydroxyethylisoquercetin (troxeisoquercetin), trishydroxyethylluteolin (troxeluteolin), α-glycosylrutin, tiliroside and the sulfates and phosphates thereof. Of the flavonoids, particular preference is given, as active substances according to the invention, to rutin, tiliroside, α-glycosylrutin and troxerutin.

Of the coumaranones, preference is given to 4,6,3′,4′-tetrahydroxybenzyl-coumaranone-3.

The term chromone derivatives is preferably taken to mean certain chromen-2-one derivatives which are suitable as active ingredients for the preventative treatment of human skin and human hair against ageing processes and harmful environmental influences. At the same time, they exhibit a low irritation potential for the skin, have a positive effect on water binding in the skin, maintain or increase the elasticity of the skin and thus promote smoothing of the skin. These compounds preferably conform to the formula III

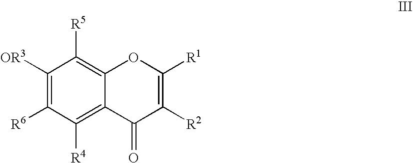

where

R¹ and R² may be identical or different and are selected from
H, —C(═O)—R⁷, —C(═O)—OR⁷
straight-chain or branched C₁- to C₂₀-alkyl groups,
straight-chain or branched C₃- to C₂₀-alkenyl groups,
straight-chain or branched C₁- to C₂₀-hydroxyalkyl groups, where the hydroxyl group may be bonded to a primary or secondary carbon atom of the chain and furthermore the alkyl chain may also be interrupted by oxygen, and/or
C₃- to C₁₀-cycloalkyl groups and/or C₃- to C₁₂-cycloalkenyl groups, where the rings may each also be bridged by -(CH₂)n- groups, where n=1 to 3,

R³ stands for H or straight-chain or branched C₁- to C₂₀-alkyl groups,

R⁴ stands for H or OR⁸,

R⁵ and R⁶ may be identical or different and are selected from
—, —OH,
straight-chain or branched C₁- to C₂₀-alkyl groups,
straight-chain or branched C₃- to C₂₀-alkenyl groups,
straight-chain or branched C₁- to C₂₀-hydroxyalkyl groups, where the hydroxyl group may be bonded to a primary or secondary carbon atom of the chain and furthermore the alkyl chain may also be interrupted by oxygen and

R⁷ stands for H, straight-chain or branched C₁- to C₂₀-alkyl groups, a polyhydroxyl compound, such as preferably an ascorbic acid radical or glycosidic radicals, and

R⁸ stands for H or straight-chain or branched C₁- to C₂₀-alkyl groups, where at least 2 of the substituents R¹, R², R⁴-R⁶ are not H or at least one substituent from R¹ and R² stands for —C(═O)—R⁷ or —C(═O)—OR⁷.

The proportion of one or more compounds selected from flavonoids, chromone derivatives and coumaranones in the composition according to the invention is preferably from 0.001 to 5% by weight, particularly preferably from 0.01 to 2% by weight, based on the composition as a whole.

The compositions having light-protection properties according to the invention may in addition comprise further conventional skin-protecting or skincare active ingredients. These can in principle be any active ingredients known to the person skilled in the art.

Particularly preferred active ingredients are pyrimidinecarboxylic acids and/or aryl oximes.

Pyrimidinecarboxylic acids occur in halophilic microorganisms and play a role in osmoregulation of these organisms (E. A. Galinski et al., Eur. J. Biochem., 149 (1985) pages 135-139). Of the pyrimidinecarboxylic acids, particular mention should be made here of ectoine ((S)-1,4,5,6-tetrahydro-2-methyl4-pyrimidinecarboxylic acid) and hydroxyectoine ((S,S)1,4,5,6-tetrahydro-5-hydroxy-2-methyl4-pyrimidinecarboxylic acid) and derivatives thereof. These compounds stabilise enzymes and other biomolecules in aqueous solutions and organic solvents. Furthermore, they stabilise, in particular, enzymes against denaturing conditions, such as salts, extreme pH values, surfactants, urea, guanidinium chloride and other compounds.

Ectoine and ectoine derivatives, such as hydroxyectoine, can advantageously be used in medicaments. In particular, hydroxyectoine can be employed for the preparation of a medicament for the treatment of skin diseases. Other areas of application of hydroxyectoine and other ectoine derivatives are typically in areas in which, for example, trehalose is used as additive. Thus, ectoine derivatives, such as hydroxyectoine, can be used as protectant in dried yeast and bacteria cells. Pharmaceutical products, such as non-glycosylated, pharmaceutical active peptides and proteins, for example t-PA, can also be protected with ectoine or its derivatives.

Of the cosmetic applications, particular mention should be made of the use of ectoine and ectoine derivatives for the care of aged, dry or irritated skin. Thus, European patent application EP-A-0 671 161 describes, in particular, that ectoine and hydroxyectoine are employed in cosmetic compositions, such as powders, soaps, surfactant-containing cleansing products, lipsticks, rouge, make-up, care creams and sunscreen preparations.

Preference is given here to the use of a pyrimidinecarboxylic acid of the following formula IV

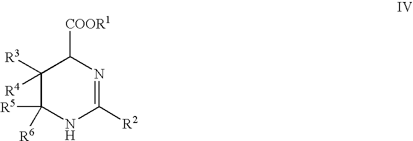

in which R¹ is a radical H or C1-8-alkyl, R² is a radical H or C1-4-alkyl, and R³, R⁴, R⁵ and R⁶ are each, independently of one another, a radical from the group H, OH, NH₂ and C1-4-alkyl. Preference is given to the use of pyrimidinecarboxylic acids in which R² is a methyl or ethyl group, and R¹ or R⁵ and R⁶ are H. Particular preference is given to the use of the pyrimidine-carboxylic acids ectoine ((S)-1,4,5,6-tetrahydro-2-methyl-4-pyrimidine-carboxylic acid) and hydroxyectoine ((S,S)-1,4,5,6-tetrahydro-5-hydroxy-2-methyl-4-pyrimidinecarboxylic acid). In this case, the compositions according to the invention preferably comprise pyrimidinecarboxylic acids of this type in amounts of up to 15% by weight.

Of the aryl oximes, preference is given to the use of 2-hydroxy-5-methyllaurophenone oxime, which is also known as HMLO, LPO or F5. Its suitability for use in cosmetic compositions is disclosed, for example, in DE-A-41 16 123. Compositions which comprise 2-hydroxy-5-methyllaurophenone oxime are accordingly suitable for the treatment of skin diseases which are accompanied by inflammation. It is known that compositions of this type can be used, for example, for the therapy of psoriasis, various forms of eczema, irritative and toxic dermatitis, UV dermatitis and further allergic and/or inflammatory diseases of the skin and integumentary appendages. Compositions according to the invention which comprise aryl oximes, preferably 2-hydroxy-5-methyllaurophenone oxime, exhibit surprising antiinflammatory suitability. The compositions here preferably comprise 0.01 to 10% by weight of the aryl oxime, it being particularly preferred for the composition to comprise 0.05 to 5% by weight of aryl oxime.

All compounds or components described here that can be used in the compositions are either known and commercially available or can be synthesised by known processes.

Besides the compounds described here, the compositions according to the invention may also comprise at least one photostabiliser, preferably conforming to the formula V

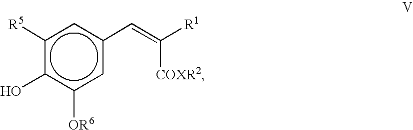

where
R¹ is selected from —C(O)CH₃, —CO₂R³, —C(O)NH₂ and —C(O)N(R⁴)₂;
X is O or NH;
R² stands for a linear or branched C₁₋₃₀-alkyl radical;
R³ stands for a linear or branched C₁₋₂₀-alkyl radical;
all R⁴,independently of one another, stand for H or linear or branched C₁₋₈-alkyl radicals;
R⁵ stands for H, a linear or branched C₁₋₈-alkyl radical or a linear or branched —O—C₁₋₈-alkyl radical; and
R⁶ stands for a C₁₋₈-alkyl radical,

where the photostabiliser is particularly preferably bis(2-ethylhexyl) 2-(4-hydroxy-3,5-dimethoxybenzylidene)malonate. Corresponding photostabilisers and their preparation and use are described in International patent application WO 03/007906, the disclosure content of which expressly also belongs to the subject-matter of the present application.

The compositions according to the invention can be prepared by processes which are well known to the person skilled in the art, in particular by the processes which serve for the preparation of oil-in-water emulsions or water-in-oil emulsions.

The present invention furthermore relates to compositions comprising the particles according to the invention and one or more cosmetically, pharmaceutically and/or dermatologically suitable carriers, a process for the preparation of a composition which is characterised in that particles according to the invention are mixed with a cosmetically, pharmaceutically and/or dermatologically suitable carrier, and the use of particles for the preparation of a composition.

These compositions can be, in particular, in the form of simple or complex emulsions (O/W, W/O, O/W/O or W/O/W), such as creams, milks, gels or gel creams, powders and solid sticks, and they may, if desired, be formulated as aerosols and be in the form of foams or sprays. These compositions are preferably in the form of an O/W emulsion.

The cosmetic compositions according to the invention can be used as compositions for protection of the human epidermis or of the hair against UV radiation, as sunscreen compositions or make-up products.

It should be pointed out that in the formulations according to the invention for sun protection which have a carrier of the oil-in-water emulsion type, the aqueous phase (which comprises, in particular, the hydrophilic filters) generally makes up 50 to 95% by weight and preferably 70 to 90% by weight, based on the formulation as a whole, the oil phase (which comprises, in particular, the lipophilic filters) makes up 5 to 50% by weight and preferably 10 to 30% by weight, based on the formulation as a whole, and the (co)emulsifier or (co)emulsifiers make(s) up 0.5 to 20% by weight and preferably 2 to 10% by weight, based on the formulation as a whole.

Suitable compositions are those for external use, for example in the form of a cream, lotion or gel or as a solution which can be sprayed onto the skin. Suitable for internal use are administration forms such as capsules, coated tablets, powders, tablet solutions or solutions.

Examples which may be mentioned of application forms of the compositions according to the invention are: solutions, suspensions, emulsions, PIT emulsions, pastes, ointments, gels, creams,,lotions, powders, soaps, surfactant-containing cleansing preparations, oils, aerosols and sprays.

Examples of other application forms are sticks, shampoos and shower products. Any desired customary carriers, auxiliaries and, if desired, further active ingredients may be added to the composition.

Preferred auxiliaries originate from the group of the preservatives, antioxidants, stabilisers, solubilisers, vitamins, colorants and odour improvers.

Ointments, pastes, creams and gels may comprise the customary carriers, for example animal and vegetable fats, waxes, paraffins, starch, tragacanth, cellulose derivatives, polyethylene glycols, silicones, bentonites, silica, talc and zinc oxide, or mixtures of these substances.

Powders and sprays may comprise the customary carriers, for example lactose, talc, silica, aluminium hydroxide, calcium silicate and polyamide powder, or mixtures of these substances. Sprays may additionally comprise the customary propellants, for example chlorofluorocarbons, propane/butane or dimethyl ether.

Solutions and emulsions may comprise the customary carriers, such as solvents, solubilisers and emulsifiers, for example water, ethanol, isopropanol, ethyl carbonate, ethyl acetate, benzyl alcohol, benzyl benzoate, propylene glycol, 1,3-butyl glycol, oils, in particular cottonseed oil, peanut oil, corn oil, olive oil, castor oil and sesame oil, glycerol fatty acid esters, polyethylene glycols and fatty acid esters of sorbitan, or mixtures of these substances.

Suspensions may comprise the customary carriers, such as liquid diluents, for example water, ethanol or propylene glycol, suspending agents, for example ethoxylated isostearyl alcohols, polyoxyethylene sorbitol esters and polyoxyethylene sorbitan esters, microcrystalline cellulose, aluminium metahydroxide, bentonite, agar-agar and tragacanth, or mixtures of these substances.

Soaps may comprise the customary carriers, such as alkali metal salts of fatty acids, salts of fatty acid monoesters, fatty acid protein hydrolysates, isethionates, lanolin, fatty alcohol, vegetable oils, plant extracts, glycerol, sugars, or mixtures of these substances.

Surfactant-containing cleansing products may comprise the customary carriers, such as salts of fatty alcohol sulfates, fatty alcohol ether sulfates, sulfosuccinic acid monoesters, fatty acid protein hydrolysates, isethionates, imidazolinium derivatives, methyl taurates, sarcosinates, fatty acid amide ether sulfates, alkylamidobetaines, fatty alcohols, fatty acid glycerides, fatty acid diethanolamides, vegetable and synthetic oils, lanolin derivatives, ethoxylated glycerol fatty acid esters, or mixtures of these substances.

Face and body oils may comprise the customary carriers, such as synthetic oils, such as fatty acid esters, fatty alcohols, silicone oils, natural oils, such as vegetable oils and oily plant extracts, paraffin oils, lanolin oils, or mixtures of these substances.

Further typical cosmetic application forms are also lipsticks, lip-care sticks, mascara, eyeliner, eye shadow, rouge, powder make-up, emulsion make-up and wax make-up, and sunscreen, pre-sun and after-sun preparations.

The preferred composition forms according to the invention include, in particular, emulsions.

Emulsions according to the invention are advantageous and comprise, for example, the said fats, oils, waxes and other fatty bodies, as well as water and an emulsifier, as usually used for a composition of this type.

The lipid phase may advantageously be selected from the following group of substances:
mineral oils, mineral waxes
oils, such as triglycerides of capric or caprylic acid, furthermore natural oils, such as, for example, castor oil;
fats, waxes and other natural and synthetic fatty bodies, preferably esters of fatty acids with alcohols having a low C number, for example with isopropanol, propylene glycol or glycerol, or esters of fatty alcohols with alkanoic acids having a low C number or with fatty acids;
silicone oils, such as, for example, dimethylpolysiloxanes, diethylpolysiloxanes, diphenylpolysiloxanes and mixed forms thereof.

For the purposes of the present invention, the oil phase of the emulsions, oleogels or hydrodispersions or lipodispersions is advantageously selected from the group of the esters of saturated and/or unsaturated, branched and/or unbranched alkanecarboxylic acids having a chain length of 3 to 30 C atoms and saturated and/or unsaturated, branched and/or unbranched alcohols having a chain length of 3 to 30 C atoms, or from the group of the esters of aromatic carboxylic acids and saturated and/or unsaturated, branched and/or unbranched alcohols having a chain length of 3 to 30 C atoms. Ester oils of this type can then advantageously be selected from the group isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and synthetic, semi-synthetic and natural mixtures of esters of this type, for example jojoba oil.

The oil phase may furthermore advantageously be selected from the group of the branched and unbranched hydrocarbons and waxes, silicone oils, dialkyl ethers, or the group of the saturated or unsaturated, branched or unbranched alcohols, and fatty acid triglycerides, specifically the triglycerol esters of saturated and/or unsaturated, branched and/or unbranched alkanecarboxylic acids having a chain length of 8 to 24, in particular 12-18 C atoms. The fatty acid triglycerides may advantageously be selected, for example, from the group of the synthetic, semi-synthetic and natural oils, for example olive oil, sunflower oil, soya oil, peanut oil, rapeseed oil, almond oil, palm oil, coconut oil, palm kernel oil and the like.

Any desired mixtures of oil and wax components of this type may also advantageously be employed for the purposes of the present invention. It may also be advantageous to employ waxes, for example cetyl palmitate, as the only lipid component of the oil phase.

The oil phase is advantageously selected from the group 2-ethylhexyl isostearate, octyldodecanol, isotridecyl isononanoate, isoeicosane, 2-ethylhexyl cocoate, C₁₂₋₁₅-alkyl benzoate, caprylic/capric acid triglyceride and dicapryl ether.

Particularly advantageous are mixtures of C₁₂-₁₅-alkyl benzoate and 2-ethylhexyl isostearate, mixtures of C₁₂₋₁₅-alkyl benzoate and isotridecyl isononanoate, as well as mixtures of C₁₂₋₁₅-alkyl benzoate, 2-ethylhexyl isostearate and isotridecyl isononanoate.

Of the hydrocarbons, paraffin oil, squalane and squalene may advantageously be used for the purposes of the present invention.

Furthermore, the oil phase may also advantageously have a content of cyclic or linear silicone oils or consist entirely of oils of this type, although it is preferred to use an additional content of other oil-phase components in. addition to the silicone oil or the silicone oils.

The silicone oil to be used in accordance with the invention is advantageously cyclomethicone (octamethylcyclotetrasiloxane). However, it is also advantageous for the purposes of the present invention to use other silicone oils, for example hexamethylcyclotrisiloxane, polydimethylsiloxane, poly(methylphenylsiloxane).

Also particularly advantageous are mixtures of cyclomethicone and isotridecyl isononanoate and of cyclomethicone and 2-ethylhexyl isostearate.

The aqueous phase of the compositions according to the invention optionally advantageously comprises alcohols, diols or polyols having a low C number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, furthermore alcohols having a low C number, for example ethanol, isopropanol, 1,2-propanediol, glycerol, and, in particular, one or more thickeners, which may advantageously be selected from the group silicon dioxide, aluminium silicates, polysaccharides and derivatives thereof, for example hyaluronic acid, xanthan gum, hydroxypropylmethylcellulose, particularly advantageously from the group of the polyacrylates, preferably a polyacrylate from the group of the so-called Carbopols, for example Carbopol grades 980, 981, 1382, 2984, 5984, in each case individually or in combination.

In particular, mixtures of the above-mentioned solvents are used. In the case of alcoholic solvents, water may be a further constituent.

Emulsions according to the invention are advantageous and comprise, for example, the said fats, oils, waxes and other fatty bodies, as well as water and an emulsifier, as usually used for a formulation of this type.

In a preferred embodiment, the compositions according to the invention comprise hydrophilic surfactants.

The hydrophilic surfactants are preferably selected from the group of the alkylglucosides, acyl lactylates, betaines and coconut amphoacetates.

The alkylglucosides are themselves advantageously selected from the group of the alkylglucosides which are distinguished by the structural formula

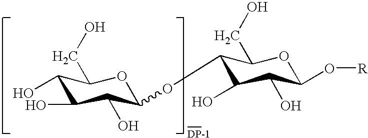

where R represents a branched or unbranched alkyl radical having 4 to 24 carbon atoms and where D̅P̅ denotes a mean degree of glucosylation of up to 2.

The value D̅P̅ represents the degree of glucosidation of the alkylglucosides used in accordance with the invention and is defined as

$$\overline{DP} = \frac{{p}_{1}}{100} \cdot 1 + \frac{{p}_{2}}{100} \cdot 2 + \frac{{p}_{3}}{100} \cdot 3 + … = \sum \frac{{p}_{i}}{100} \cdot i$$

in which p₁, p₂, p₃ to pᵢ represent the proportions of mono-, di-, tri- to i-fold glucosylated products in percent by weight. Products having degrees of glucosylation of 1-2, particularly advantageously of 1.1 to 1.5, very particularly advantageously of 1.2-1.4, in particular of 1.3, are advantageously selected in accordance with the invention.

The value D̅P̅ takes into account the fact that alkylglucosides are generally, as a consequence of their preparation, in the form of mixtures of mono- and oligoglucosides. A relatively high content of monoglucosides, typically in the order of 40-70% by weight, is advantageous in accordance with the invention.

Alkylglucosides which are particularly advantageously used in accordance with the invention are selected from the group octyl glucopyranoside, nonyl glucopyranoside, decyl glucopyranoside, undecyl glucopyranoside, dodecyl glucopyranoside, tetradecyl glucopyranoside and hexadecyl glucopyranoside.

It is likewise advantageous to employ natural or synthetic raw materials and auxiliaries or mixtures which are distinguished by an effective content of the active ingredients used in accordance with the invention, for example Plantaren® 1200 (Henkel KGaA), Oramix® NS 10 (Seppic).

The acyllactylates are themselves advantageously selected from the group of the substances which are distinguished by the structural formula

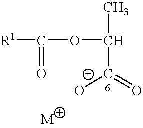

where R¹ denotes a branched or unbranched alkyl radical having 1 to 30 carbon atoms and M⁺ is selected from the group of the alkali metal ions and the group of the ammonium ions which are substituted by one or more alkyl and/or by one or more hydroxyalkyl radicals, or corresponds to half an equivalent of an alkaline earth metal ion.

For example, sodium isostearyl lactylate, for example the product Pathionic® ISL from the American Ingredients Company, is advantageous.

The betaines are advantageously selected from the group of the substances which are distinguished by the structural formula

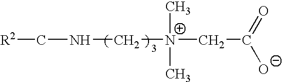

where R² denotes a branched or unbranched alkyl radical having 1 to 30 carbon atoms.

R² particularly advantageously denotes a branched or unbranched alkyl radical having 6 to 12 carbon atoms.

For example, capramidopropylbetaine, for example the product Tego® betaine 810 from Th. Goldschmidt AG, is advantageous.

A coconut amphoacetate which is advantageously selected in accordance with the invention is, for example, sodium coconut amphoacetate, as available under the name Miranol® Ultra C32 from Miranol Chemical Corp.

The compositions according to the invention are advantageously characterised in that the hydrophilic surfactant(s) is (are) present in concentrations of 0.01-20% by weight, preferably 0.05-10% by weight, particularly preferably 0.1-5% by weight, in each case based on the total weight of the composition.

For use, the cosmetic and dermatological compositions according to the invention are applied to the skin and/or the hair in an adequate amount in the usual manner for cosmetics.

Cosmetic and dermatological compositions according to the invention may exist in various forms. Thus, they can be, for example, a solution, a water-free composition, an emulsion or microemulsion of the water-in-oil (W/O) type or of the oil-in-water (O/W) type, a multiple emulsion, for example of the water-in-oil-in-water (W/O/W) type, a gel, a solid stick, an ointment or an aerosol. It is also advantageous to administer ectoines in encapsulated form, for example in collagen matrices and other conventional encapsulation materials, for example as cellulose encapsulations, in gelatine, wax matrices or liposomally encapsulated. In particular, wax matrices, as described in DE-A 43 08 282, have proven favourable. Preference is given to emulsions. O/W emulsions are particularly preferred. Emulsions, W/O emulsions and O/W emulsions are obtainable in a conventional manner.

Emulsifiers that can be used are, for example, the known W/O and O/W emulsifiers. It is advantageous to use further conventional co-emulsifiers in the preferred O/W emulsions according to the invention.

An emulsifier that has proven to be particularly preferred in accordance with the invention for O/W emulsions is the commercial product Ceralution C from Sasol.

Co-emulsifiers which are advantageously selcted in accordance with the invention are, for example, O/W emulsifiers, principally from the group of the substances having HLB values of 11-16, very particularly advantageously having HLB values of 14.5-15.5, so long as the O/W emulsifiers have saturated radicals R and R′. If the O/W emulsifiers have unsaturated radicals R and/or R′ or if isoalkyl derivatives are present, the preferred HLB value of such emulsifiers may also be lower or higher.

It is advantageous to select the fatty alcohol ethoxylates from the group of the ethoxylated stearyl alcohols, cetyl alcohols, cetylstearyl alcohols (cetearyl alcohols). Particular preference is given to the following: polyethylene glycol (13) stearyl ether (steareth-13), polyethylene glycol (14) stearyl ether (steareth-14), polyethylene glycol (15) stearyl ether (steareth-15), polyethylene glycol (16) stearyl ether (steareth16), polyethylene glycol (17) stearyl ether (steareth-17), polyethylene glycol (18) stearyl ether (steareth-18), polyethylene glycol (19) stearyl ether (steareth-19), polyethylene glycol (20) stearyl ether (steareth-20), polyethylene glycol (12) isostearyl ether (isosteareth-12), polyethylene glycol (13) isostearyl ether (isosteareth-13), polyethylene glycol (14) isostearyl ether (isosteareth-14), polyethylene glycol (15) isostearyl ether (isosteareth-1 5), polyethylene glycol (16) isostearyl ether (isosteareth-16), polyethylene glycol (17) isostearyl ether (isosteareth-17), polyethylene glycol (18) isostearyl ether (isosteareth-18), polyethylene glycol (19) isostearyl ether (isosteareth19), polyethylene glycol (20) isostearyl ether (isosteareth-20), polyethylene glycol (13) cetyl ether (ceteth-13), polyethylene glycol (14) cetyl ether (ceteth-14), polyethylene glycol (15) cetyl ether (ceteth-15), polyethylene glycol (16) cetyl ether (ceteth-16), polyethylene glycol (17) cetyl ether (ceteth-17), polyethylene glycol (18) cetyl ether (ceteth-18), polyethylene glycol (19) cetyl ether (ceteth-19), polyethylene glycol (20) cetyl ether (ceteth-20), polyethylene glycol (13) isocetyl ether (isoceteth-13), polyethylene glycol (14) isocetyl ether (isoceteth-14), polyethylene glycol (15) isocetyl ether (isoceteth-15), polyethylene glycol (16) isocetyl ether (isoceteth-16), polyethylene glycol (17) isocetyl ether (isoceteth-17), polyethylene glycol (18) isocetyl ether (isoceteth-18), polyethylene glycol (19) isocetyl ether (isoceteth-19), polyethylene glycol (20) isocetyl ether (isoceteth-20), polyethylene glycol (12) oleyl ether (oleth12), polyethylene glycol (13) oleyl ether (oleth-13), polyethylene glycol (14) oleyl ether (oleth-14), polyethylene glycol (15) oleyl ether (oleth-15), polyethylene glycol (12) lauryl ether (laureth-12), polyethylene glycol (12) isolauryl ether (isolaureth-12), polyethylene glycol (13) cetylstearyl ether (ceteareth13), polyethylene glycol (14) cetylstearyl ether (ceteareth-14), polyethylene glycol (15) cetylstearyl ether (ceteareth-15), polyethylene glycol (16) cetylstearyl ether (ceteareth-16), polyethylene glycol (17) cetylstearyl ether (ceteareth17), polyethylene glycol (18) cetylstearyl ether (ceteareth-18), polyethylene glycol (19) cetylstearyl ether (ceteareth-19), polyethylene glycol (20) cetylstearyl ether (ceteareth-20).

It is furthermore advantageous to select the fatty acid ethoxylates from the following group:

polyethylene glycol (20) stearate, polyethylene glycol (21) stearate, polyethylene glycol (22) stearate, polyethylene glycol (23) stearate, polyethylene glycol (24) stearate, polyethylene glycol (25) stearate, polyethylene glycol (12) isostearate, polyethylene glycol (13) isostearate, polyethylene glycol (14) isostearate, polyethylene glycol (15) isostearate, polyethylene glycol (16) isostearate, polyethylene glycol (17) isostearate, polyethylene glycol (18) isostearate, polyethylene glycol (19) isostearate, polyethylene glycol (20) isostearate, polyethylene glycol (21) isostearate, polyethylene glycol (22) isostearate, polyethylene glycol (23) isostearate, polyethylene glycol (24) isostearate, polyethylene glycol (25) isostearate, polyethylene glycol (12) oleate, polyethylene glycol (13) oleate, polyethylene glycol (14) oleate, polyethylene glycol (15) oleate, polyethylene glycol (16) oleate, polyethylene glycol (17) oleate, polyethylene glycol (18) oleate, polyethylene glycol (19) oleate, polyethylene glycol (20) oleate.

An ethoxylated alkyl ether carboxylic acid or salt thereof which can advantageously be used is sodium laureth-11 carboxylate. An alkyl ether sulfate which can advantageously be used is sodium laureth-14 sulfate. An ethoxylated cholesterol derivative which can advantageously be used is polyethylene glycol (30) cholesteryl ether. Polyethylene glycol (25) soyasterol has also proven successful. Ethoxylated triglycerides which can advantageously be used are the polyethylene glycol (60) evening primrose glycerides.

It is furthermore advantageous to select the polyethylene glycol glycerol fatty acid esters from the group polyethylene glycol (20) glyceryl laurate, polyethylene glycol (21) glyceryl laurate, polyethylene glycol (22) glyceryl laurate, polyethylene glycol (23) glyceryl laurate, polyethylene glycol (6) glyceryl caprate/caprinate, polyethylene glycol (20) glyceryl oleate, polyethylene glycol (20) glyceryl isostearate, polyethylene glycol (18) glyceryl oleate/cocoate.

It is likewise favourable to select the sorbitan esters from the group polyethylene glycol (20) sorbitan monolaurate, polyethylene glycol (20) sorbitan monostearate, polyethylene glycol (20) sorbitan monoisostearate, polyethylene glycol (20) sorbitan monopalmitate, polyethylene glycol (20) sorbitan monooleate.

The following can be employed as optional W/O emulsifiers, but ones which may nevertheless be advantageous in accordance with the invention:

fatty alcohols having 8 to 30 carbon atoms, monoglycerol esters of saturated and/or unsaturated, branched and/or unbranched alkanecarboxylic acids having a chain length of 8 to 24, in particular 12-18 C atoms, diglycerol esters of saturated and/or unsaturated, branched and/or unbranched alkanecarboxylic acids having a chain length of 8 to 24, in particular 12-18 C atoms, monoglycerol ethers of saturated and/or unsaturated, branched and/or unbranched alcohols having a chain length of 8 to 24, in particular 12-18 C atoms, diglycerol ethers of saturated and/or unsaturated, branched and/or unbranched alcohols having a chain length of 8 to 24, in particular 12-18 C atoms, propylene glycol esters of saturated and/or unsaturated, branched and/or unbranched alkanecarboxylic acids having a chain length of 8 to 24, in particular 12-18 C atoms, and sorbitan esters of saturated and/or unsaturated, branched and/or unbranched alkanecarboxylic acids having a chain length of 8 to 24, in particular 12-18 C atoms.

Particularly advantageous W/O emulsifiers are glyceryl monostearate, glyceryl monoisostearate, glyceryl monomyristate, glyceryl monooleate, diglyceryl monostearate, diglyceryl monoisostearate, propylene glycol mono-stearate, propylene glycol monoisostearate, propylene glycol monocaprylate, propylene glycol monolaurate, sorbitan monoisostearate, sorbitan monolaurate, sorbitan monocaprylate, sorbitan monoisooleate, sucrose distearate, cetyl alcohol, stearyl alcohol, arachidyl alcohol, behenyl alcohol, isobehenyl alcohol, selachyl alcohol, chimyl alcohol, polyethylene glycol (2) stearyl ether (steareth-2), glyceryl monolaurate, glyceryl monocaprinate, glyceryl monocaprylate.

Compositions which are preferred in accordance with the invention are particularly suitable for protecting human skin against UV-induced ageing processes and against oxidative stress, i.e. against damage caused by free radicals, as are generated, for example, by sunlight, heat or other influences. In this connection, they are in the various administration forms usually used for this application. For example, they may, in particular, be in the form of a lotion or emulsion, such as in the form of a cream or milk (O/W, W/O, O/W/O, W/O/W), in the form of oily/alcoholic, oily/aqueous or aqueous/alcoholic gels or solutions, in the form of solid sticks or may be formulated as an aerosol.

The composition may comprise cosmetic adjuvants that are usually used in this type of composition, such as, for example, thickeners, softeners, moisturisers, surface-active agents, emulsifiers, preservatives, antifoams, perfumes, waxes, lanolin, propellants, dyes and/or pigments which colour the composition itself or the skin, and other ingredients usually used in cosmetics.

The dispersant or solubiliser used can be an oil, wax or other fatty body, a lower monoalcohol or a lower polyol or mixtures thereof. Particularly preferred monoalcohols or polyols include ethanol, i-propanol, propylene glycol, glycerol and sorbitol.

A preferred embodiment of the invention is an emulsion in the form of a protective cream or milk which, apart from the compound(s) of the formula I, comprises, for example, fatty alcohols, fatty acids, fatty acid esters, in particular triglycerides of fatty acids, lanolin, natural and synthetic oils or waxes and emulsifiers in the presence of water.

Further preferred embodiments are oily lotions based on natural or synthetic oils and waxes, lanolin, fatty acid esters, in particular triglycerides of fatty acids, or oily-alcoholic lotions based on a lower alcohol, such as ethanol, or a glycerol, such as propylene glycol, and/or a polyol, such as glycerol, and oils, waxes and fatty acid esters, such as triglycerides of fatty acids.

The composition according to the invention may also be in the form of an alcoholic gel which comprises one or more lower alcohols or polyols, such as ethanol, propylene glycol or glycerol, and a thickener, such as siliceous earth. The oily/alcoholic gels also comprise natural or synthetic oil or wax.

The solid sticks consist of natural or synthetic waxes and oils, fatty alcohols, fatty acids, fatty acid esters, lanolin and other fatty bodies.

If a composition is formulated as an aerosol, the customary propellants, such as alkanes, fluoroalkanes and chlorofluoroalkanes, are generally used.

The cosmetic composition may also be used to protect the hair against photochemical damage in order to prevent colour changes, bleaching or damage of a mechanical nature. In this case, a suitable formulation is in the form of a rinse-out shampoo, lotion, gel or emulsion, the composition in question being applied before or after shampooing, before or after colouring or bleaching or before or after permanent waving. It is also possible to select a composition in the form of a lotion or gel for styling and treating the hair, in the form of a lotion or gel for brushing or laying a water wave, in the form of a hair lacquer, permanent-waving composition, colorant or bleach for the hair. The composition having light-protection properties may comprise various adjuvants used in this type of composition, such as surface-active agents, thickeners, polymers, softeners, preservatives, foam stabilisers, electrolytes, organic solvents, silicone derivatives, oils, waxes, antigrease agents, dyes and/or pigments which colour the composition itself or the hair, or other ingredients usually used for hair care.

The following examples are intended to explain the invention in greater detail, but without restricting it.

### EXAMPLES

### Example 1

### Samples 1-4 Having Particle Diameters of 100 nm

a) Preparation of 2-hydroxy-4-(3-triethoxysilylpropoxy)diphenyl Ketone 10 drops of a 5% solution of a vinyl/siloxane complex hydrosilylation catalyst in toluene are added dropwise under a nitrogen atmosphere to a mixture of 5.08 g (0.02 mol) of 4-allyloxy-2-hydroxybenzophenone and 3.28 g (0.02 mol) of triethoxysilane in 100 ml of dry toluene. The reaction is complete after approximately 30 minutes. The solvent is removed at 50° C. under reduced pressure, giving the product as a slightly yellow viscous oil.

b) Copolymerisation

160 ml of ethanol and 83 ml of demineralised water are warmed to 58° C., and 9.4 ml of a 25% ammonia solution (in water) are added. A mixture, warmed to 58° C., of 20.94 g of tetraethoxysilane (TEOS) and 427 mg of the product obtained in step a) is added to the ethanol/water/ammonia mixture with stirring. The mixture is stirred for 15 seconds, and the reaction mixture is then left to stand at 58° C. without stirring for 2 hours. After separation from the reaction mixture, pulverulent SiO₂ monospheres with 2-hydroxy4-(3-triethoxysilylpropoxy)diphenyl ketone in the core of the particles are obtained, where the monospheres have an average diameter of 100 nm.

### Example 2

### Samples 5-6 Having Particle Diameters of 250 nm

a) See Example 1a)

b) Copolymerisation

150 ml of ethanol and 56 ml of demineralised water are warmed to 60° C., and 35 ml of a 25% ammonia solution (in water) are added. A mixture, warmed to 60° C., of 20.94 g of tetraethoxysilane (TEOS) and 427 mg of the product obtained in step a) is added to the ethanol/water/ammonia mixture with stirring. The mixture is stirred for 15 seconds, and the reaction mixture is then left to stand at 60° C. without stirring for 2 hours. After separation from the reaction mixture, pulverulent SiO₂ monospheres with 2-hydroxy-4-(3-triethoxysilylpropoxy)diphenyl ketone in the core of the particles are obtained, where the monospheres have an average diameter of 250 nm.

### Example 3

### Samples 7-8 Having Particle Diameters of 500 nm

a) See Example 1a)

b) Copolymerisation

150 ml of ethanol and 56 ml of demineralised water are warmed to 30° C., and 35 ml of a 25% ammonia solution (in water) are added. A mixture, warmed to 30° C., of 20.94 g of tetraethoxysilane (TEOS) and 427 mg of the product obtained in step a) is added to the ethanol/water/ammonia mixture with stirring. The mixture is stirred for 15 seconds, and the reaction mixture is then left to stand at 30° C. without stirring for 2 hours. After separation from the reaction mixture, pulverulent SiO₂ monospheres with 2-hydroxy-4-(3-triethoxysilylpropoxy)diphenyl ketone in the core of the particles are obtained, where the monospheres have an average diameter of 500 nm.

### Example 4

### Samples 9-10 Having Particle Diameters of 250 nm With Additional Surface Functionalisation

a) See Example 1a)

b) Copolymerisation and Additional Functionalisation

150 ml of ethanol and 56 ml of demineralised water are warmed to 60° C., and 35 ml of a 25% ammonia solution (in water) are added. A mixture, warmed to 60° C., of 20.94 g of tetraethoxysilane (TEOS) and 427 mg of the product obtained in step a) is added to the ethanol/water/ammonia mixture with stirring. The mixture is stirred for 15 seconds, and the reaction mixture is then left to stand at 60° C. without stirring for 2 hours. The reaction is subsequently initiated by further stirring, and still unreacted product obtained in step a) is brought to reaction. The mixture is stirred at 60° C. for a further 2.5 hours. After separation from the reaction mixture, pulverulent SiO₂ monospheres with 2-hydroxy4-(3-triethoxysilylpropoxy)diphenyl ketone in the core and on the surface of the particles are obtained, where the monospheres have an average diameter of 250 nm.

### Example 5

### Sample 11 Having A Particle Diameter Of 250 nm With Additional Surface Functionalisation

a) See Example 1a)

b) Copolymerisation and Additional Functionalisation

150 ml of ethanol and 56 ml of demineralised water are warmed to 60° C., and 35 ml of a 25% ammonia solution (in water) are added. A mixture, warmed to 60° C., of 20.94 g of tetraethoxysilane (TEOS) and 427 mg of the product obtained in step a) is added to the ethanol/water/ammonia mixture with stirring. The mixture is stirred for 15 seconds, and the reaction mixture is then left to stand at 60° C. without stirring for 2 hours. The mixture is subsequently stirred again, and 850 mg of the product obtained in step a) (dissolved in 100 ml of ethanol) are added dropwise to the suspension over the course of one hour. The mixture is stirred at 60° C. for a further 2 hours. After separation from the reaction mixture, pulverulent SiO₂ monospheres with 2-hydroxy4-(3-triethoxysilylpropoxy)diphenyl ketone in the core and on the surface of the particles are obtained, where the monospheres have an average diameter of 250 nm.

UV Measurements:

50 mg of the respective sample are suspended in 50 ml of 2-propanol in a 100 ml volumetric flask with the aid of an ultrasound bath. The volumetric flask is made up to 100 ml with 2-propanol, and the suspension is rehomogenised. A sample of the solution is measured in the UV spectrometer (model: Perkin Elmer Lambda 900 with Ulbricht sphere (150 mm diameter)).

The results of the UV-spectrometric investigations of the samples obtained in accordance with Examples 1 to 5 are summarised in Table 1.

TABLE 1
Overview of samples 1 to 11 and the comparative samples

| | | | Proportion of | Molar absorption |
|---|---|---|---|---|
| | Functionalisation | Average particle | org. compound | coefficient |
| Sample | site | diameter [nm] | [mmol/g] | ε [Lmol⁻¹ cm⁻¹] |
| 1 | core | 100 | 0.12 | 9588 |
| 2 | core | 100 | 0.22 | 10269 |
| 3 | core | 100 | 0.32 | 9086 |
| 4 | core | 100 | 0.41 | 8933 |
| 5 | core | 250 | 0.21 | 9628 |
| 6 | core | 250 | 0.313 | 8907 |
| 7 | core | 500 | 0.10 | 7989 |
| 8 | core | 500 | 0.21 | 8751 |
| 9 | core + surface | 250 | 0.02 | 46456 |
| 10 | core + surface | 250 | 0.18 | 10037 |
| 11 | core + surface | 250 | 0.27 | 7804 |
| Comparison 1^{[a]} | surface | 250 | 0.12^{[b]} | 6795 |
| Comparison 2^{[a]} | surface | 500 | 0.14^{[b]} | 7163 |

^{[a]}Particles inaccordance with EP 1 205 177
^{[b]}Proportion on the surface

The particles according to the invention have higher molar absorption coefficients, i.e. they have better light-protection properties, than the corresponding comparative samples in accordance with the prior art.

Photostability Measurements:

Samples are irradiated for 130 min with a power of 89.6 W/m² using a Suntest CPS with xenon lamp (radiation restricted to wavelengths≧290 nm). The UV spectra of the irradiated samples are compared with those of unirradiated samples. Table 2 summarises the results for the UV activity of the samples after irradiation.

TABLE 2
Photostability of individual samples after irradiation in (%)

| UV region | Benzophenone-3 | Sample 5 | Comparison 1^{[a]} |
|---|---|---|---|
| UV | 79 ± 3 | 93 ± 1 | 89 ± 1 |
| (290-400 nm) | | | |
| UVA | 80 ± 3 | 95 ± 1 | 88 ± 1 |
| (320-400 nm) | | | |
| UVB | 77 ± 2 | 89 ± 1 | 89 ± 2 |
| (290-320 nm) | | | |

^{[a]}Particles in accordance with EP 1 205 177

Benzophenone-3 is 80% photostable. The samples according to the invention (sample 5) exhibit improved photostability, which is also better than in the case of samples in accordance with the prior art which have been functionalised on the surface (comparison 1).

Examples of Skin-Protection Formulations

Skin-protection lotion (O/W)

| Raw material | | INCI | % by wt. | |
|---|---|---|---|---|
| A | Particles from one of | | | 5.00 |
| | Examples 1 to 5 | | | |
| | Emulsifier E 2155 | Stearyl Alcohol (and) | (2) | 3.00 |
| | | Steareth-7 (and) Steareth-10 | | |
| | Teginacid H | Glyceryl Stearate (and) | (2) | 3.00 |
| | | Ceteth-20 | | |
| | Imwitor 900 | Glyceryl Stearate | (4) | 3.00 |
| | Lunacera M | Microwax | (6) | 1.00 |
| | Luvitol EHO | Cetearyl Octanoate | (3) | 11.50 |
| | Cetiol | Oleyl Oleate | (5) | 6.00 |
| | Miglyol 812 neutral oil | Caprylic/Capric | (4) | 6.00 |
| | | Triglyceride | | |
| B | Propane-1,2-diol | Propylene Glycol | (1) | 4.00 |
| | Allantoin | Allantoin | (1) | 0.20 |
| | Preservatives | | | q.s. |
| | Water, demineralised | Aqua | | to 100.00 |

Preparation:

Phase A is warmed to 75° C. and phase B to 80° C. Phase B is slowly added to phase A with stirring. The mixture is homogenised and cooled with stirring.

Notes:

Viscosity 9200 mPas (Brookfield RVT, sp. C, 10 rpm) at 24° C.

pH₂₄°C=5.0

Preservatives:

0.05% of propyl 4-hydroxybenzoate

0.15% of methyl 4-hydroxybenzoate

Sources of Supply:

(1) Merck KGaA, Darmstadt

(2) Th. Goldschmidt, Essen

(3) BASF, Ludwigshafen

(4) Hüls, Troisdorf AG, Witten

(5) Henkel, Dujsseldorf

(6) H.B. Fuller GmbH, Luineburg

Skin-protection lotion (O/W)

| Raw material | | INCI | % by wt. | |
|---|---|---|---|---|
| A | Particles from one of | | | 1.00 |
| | Examples 1 to 5 | | | |
| | Emulsifier E 2155 | Stearyl Alcohol (and) | (2) | 3.00 |
| | | Steareth-7 (and) Steareth-10 | | |
| | Teginacid H | Glyceryl Stearate (and) | (2) | 3.00 |
| | | Ceteth-20 | | |
| | Imwitor 900 | Glyceryl Stearate | (4) | 3.00 |
| | Lunacera M | Microwax | (6) | 1.00 |
| | Luvitol EHO | Cetearyl Octanoate | (3) | 11.50 |
| | Cetiol | Oleyl Oleate | (5) | 7.00 |
| | Miglyol 812 neutral oil | Caprylic/Capric | (4) | 7.00 |
| | | Triglyceride | | |
| B | Propane-1,2-diol | Propylene Glycol | (1) | 4.00 |
| | Allantoin | Allantoin | (1) | 0.20 |
| | Preservatives | | | q.s. |
| | Water, demineralised | Aqua | | to 100.00 |

Preparation:

Phase A is warmed to 75° C. and phase B to 80° C. Phase B is slowly added to phase A with stirring. The mixture is homogenised and cooled with stirring.

Notes:

Preservatives:

0.05% of propyl 4-hydroxybenzoate

0. 15% of methyl 4-hydroxybenzoate

Sources of supply:

(1) Merck KGaA, Darmstadt

(2) Th. Goldschmidt, Essen

(3) BASF, Ludwigshafen

(4) Hüls, Troisdorf AG, Witten

(5) Henkel, Dusseldorf

(6) H.B. Fuller GmbH, Luïneburg

Examples of Sunscreen Formulations

Sunscreen lotion comprising IR3535 ™ (O/W)

| Raw material | | INCI | % by wt. | |
|---|---|---|---|---|
| A | Particles from one | | | 3.00 |
| | of Examples | | | |
| | 1 to 5 | | | |
| | Eusolex 6300 | 4-Methylbenzylidene Camphor | (1) | 3.00 |
| | IR 3535 ™ | Ethyl | (1) | 10.00 |
| | | Butylacetylaminopropionate | | |
| | (−)-?-Bisabolol | Bisabolol | (1) | 0.30 |
| | Montanov 68 | Cetearyl Alcohol (and) | (2) | 4.00 |
| | | Cetearyl Glucoside | | |
| | Myritol 312 | Caprylic/Capric Triglyceride | (3) | 2.00 |
| | Mirasil CM 5 | Cyclomethicone | (4) | 2.00 |
| | Mirasil DM 350 | Dimethicone | (4) | 1.00 |
| B | Water, | Aqua | | to 100 |
| | demineralised | | | |
| | Glycerol, 87% | Glycerin | (1) | 3.00 |
| | Preservatives | | | q.s. |
| C | Rhodicare S | Xanthan Gum | (4) | 0.50 |

Preparation:

Phases A and B are warmed to 75° C. separately from one another. Phase C is slowly added to phase B at 75° C. with stirring. The mixture is stirred until it is homogeneous. Phase A is subsequently added to the mixture. The mixture is stirred until it is homogeneous, and then cooled with stirring.

Notes:

Preservatives:

0.05% of propyl 4-hydroxybenzoate

0.15% of methyl 4-hydroxybenzoate

0.30% of Germall 115 (ISP, Frechen)

Sources of Supply:

(1) Merck KGaA, Darmstadt

(2) Interorgana, Cologne

(3) Henkel, KGaA, Duïsseldorf

(4) Rhodia, Frankfurt

Sunscreen milk (O/W)

| Raw material | | INCI | % by wt. | |
|---|---|---|---|---|
| A | Particles from | | | 4.00 |
| | one of Examples | | | |
| | 1 to 5 | | | |
| | Eusolex 6300 | 4-Methylbenzylidene | (1) | 4.00 |
| | | Camphor | | |
| | Emulsifier E 2155 | Stearyl Alcohol (and) | (2) | 3.00 |
| | | Steareth-7 (and) | | |
| | | Steareth-10 | | |
| | Teginacid H | Glyceryl Stearate (and) | (2) | 2.00 |
| | | Ceteth-20 | | |
| | Luvitol EHO | Cetearyl Octanoate | (3) | 14.00 |
| | Imwitor 900 | Glyceryl Stearate | (4) | 3.00 |
| | Cetiol | Oleyl Oleate | (5) | 6.00 |
| | Lunacera M | Microwax | (6) | 1.00 |
| | Miglyol 812 neutral | Caprylic/Capric | (4) | 4.00 |
| | oil | Triglyceride | | |
| B | Eusolex 232 | Phenylbenzimidazole | (1) | 2.00 |
| | | Sulfonic Acid | | |
| | Tris(hydroxymethyl)- | Tromethamine | (1) | 1.07 |
| | aminomethane | | | |
| | Propane-1,2-diol | Propylene Glycol | (1) | 4.00 |
| | Allantoin | Allantoin | (1) | 0.20 |
| | Preservatives | | | q.s. |
| | Water, demineralised | Aqua | | to 100.00 |
| C | Carbopol ETD 2050 | Carbomer ETD 2050 | (7) | 0.25 |
| | Water, demineralised | Aqua | | 30.00 |
| D | Tris(hydroxymethyl)- | Tromethamine | (1) | 0.25 |
| | aminomethane | | | |
| | Water, demineralised | Aqua | | 4.00 |

Preparation:

Carbopol ETD 2050 is homogeneously dispersed in water in order to obtain phase C. Phase D is then incorporated into phase C with homogenisation. For the neutralisation of Eusolex 232, the tris(hydroxymethyl)aminomethane is dissolved in the water of phase B, and Eusolex 232 is added with stirring. After complete dissolution, the remaining ingredients of phase B are added, and phase B is slowly incorporated into phases C/D with homogenisation. Phase A is dissolved with heating and slowly added with homogenisation.

Notes:

Preservatives:

0.05% of propyl 4-hydroxybenzoate

0.15% of methyl 4-hydroxybenzoate

Sources of Supply:

(1) Merck KGAA, Darmstadt

(2) Th. Goldschmidt, Essen

(3) BASF, Ludwigshafen

(4) Hüls, Troisdorf AG, Witten

(5) Henkel, Düsseldorf

(6) H. B. Fuller GmbH, Luneburg

(7) Goodrich, Neuss

Sunscreen lotion (W/O)

| Raw material | | | % by wt. |
|---|---|---|---|
| A | Particles from one of | (1) | 5.00 |
| | Examples 1 to 5 | | |
| | Eusolex HMS | (1) | 5.00 |
| | Eusolex OS | (1) | 5.00 |
| | Eusolex OCR | (1) | 5.00 |
| | Abil WE 09 | (2) | 5.00 |
| | Jojoba oil | (3) | 3.00 |
| | Cetiol V | (4) | 3.00 |
| | Prisorine 2021 | (5) | 2.00 |
| | Lunacera M | (6) | 1.80 |
| | Miglyol 812 neutral oil | (6) | 3.00 |
| B | Glycerol (about 87%) | (1) | 2.00 |
| | Sodium chloride | (1) | 0.40 |
| | Preservatives | (1) | q.s. |
| | Water, demineralised | | to 100.00 |

Preparation:

Phase B is warmed to 80° C. and Phase A to 75° C. Phase B is slowly stirred into phase A. The mixture is homogenised and cooled with stirring.

Notes:

Preservatives:

0.05% of propyl 4-hydroxybenzoate

0.15% of methyl 4-hydroxybenzoate

Sources of supply:

(1) Merck KGaA, Darmstadt

(1) Th. Goldschmidt A G, Essen

(2) H. Lamotte, Bremen

(3) Henkel KGaA, Duisseldorf

(4) Unichema, Emmerich

(5) H. B. Fuller, Luineburg

(6) Hüls Troisdorf A G, Witten

Sunscreen cream (W/O)

| Raw material | | INCI | % by wt. | |
|---|---|---|---|---|
| A | Particles from one of | | (1) | 2.50 |
| | Examples 1 to 5 | | | |
| | Eusolex T-2000 | Micronised Titanium | (1) | 2.50 |
| | | Dioxide | | |
| | Eusolex 6300 | 4-Methylbenzylidene | (1) | 2.00 |
| | | Camphor | | |
| | Dehymuls E | Dicocoyl | (2) | 6.00 |
| | | Pentyerythrityl | | |
| | | Citrate (and) Sorbitan | | |
| | | Sesquioleate (and) | | |
| | | Cera Alba (and) | | |
| | | Aluminium Stearate | | |
| | Arlacel 989 | PEG-7 Hydrogenated | (3) | 1.00 |
| | | Castor Oil | | |
| | Beeswax | Cera Alba | (1) | 2.00 |
| | Zinc stearate | Zinc Stearate | (1) | 2.00 |
| | Cetiol J 600 | Oleyl Erucate | (2) | 6.00 |
| | Cetiol V | Decyl Oleate | (2) | 6.00 |
| | Cetiol OE | Dicaprylyl Ether | (2) | 5.00 |
| | Dow Corning 200 | Dimethicone | (4) | 1.00 |
| | (100 cs) | | | |
| | DL-?-tocopherol acetate | Tocopheryl Acetate | (1) | 1.00 |
| | Vitamin A palmitate | Retinyl Palmitate | (5) | 0.50 |
| B | Eusolex 232 | Phenylbenzimidazole | (1) | 2.00 |
| | | Sulfonic Acid | | |
| | Tris(hydroxymethyl)- | Tromethamine | (1) | 0.88 |
| | aminomethane | | | |
| | Glycerol (about 87%) | Glycerin | (1) | 5.00 |
| | Magnesium sulfate | Magnesium Sulfate | (1) | 1.00 |
| | hepta-hydrate | | | |
| | Allantoin | Allantoin | (1) | 0.20 |
| | Preservatives | | | q.s. |
| | Water, demineralised | | | to 100.00 |

Preparation:

For the neutralisation of Eusolex 232, the tris(hydroxymethyl)aminomethane is dissolved in the water of phase B, and Eusolex 232 is added with stirring. After complete dissolution, the remaining raw materials of phase B are added and warmed to 80° C. Phase A is warmed to 75° C. Phase B is slowly stirred into phase A, and the mixture is cooled with stirring.

Notes:

Preservatives:

0.05% of propyl 4-hydroxybenzoate

0.15% of methyl 4-hydroxybenzoate

Sources of Supply:

(1) Merck KGaA, Darmstadt

(2) Henkel KGBA, Düsseldorf

(3) ICI, Essen

(4) Dow Corning, Düsseldorf

(5) Hoffmann La Roche, Switzerland

Sunscreen gel (O/W) with UV-A/B protection

| Raw material | | | % by wt. |
|---|---|---|---|
| A | Particles from one of Examples 1 to 5 | (1) | 2.00 |
| | Eusolex 2292 | (1) | 5.50 |
| | Oxynex K liquid | (1) | 1.00 |
| | Luvitol EHO | (2) | 9.00 |
| | Dow Corning 200 (100 cs) | (3) | 2.00 |
| | Antaron V-220 | (4) | 2.00 |
| | Jojoba oil | (5) | 5.00 |
| B | Tris(hydroxymethyl)aminomethane | (1) | 0.60 |
| | Preservatives | | q.s. |
| | Water, demineralised | | to 100.00 |
| C | Pemulen TR-1 | (6) | 0.50 |
| | Water, demineralised | | 29.50 |
| D | Aloe Vera Gel 1:10 | (7) | 1.00 |

Preparation:

The Pemulen TR-1 is homogeneously dispersed in water and pre-swollen in order to obtain phase C. Phase B is incorporated into phase C with homogenisation. Phase A is dissolved with warming and slowly added with homogenisation. Phase D is added at 35° C. and again homogenised.

Notes:

Preservatives:

0.05% of propyl 4-hydroxybenzoate

0.15% of methyl 4-hydroxybenzoate

Sources of Supply:

(1) Merck KGaA, Darmstadt

(2) BASF, Ludwigshafen

(3) Dow Corning, Düsseldorf

(4) GAF, Frechen

(5) Henry Lamotte, Bremen

(6) Goodrich, Neuss

(7) Galke, Gittelde

Sunscreen spray (O/W)

| Raw material | | INCI | % by wt. | |
|---|---|---|---|---|
| A | Particles from one of | | (1) | 3.00 |
| | Examples 1 to 5 | | | |
| | Eusolex 2292 | Octyl Methoxycinnamate | (1) | 7.50 |
| | Eusolex HMS | Homosalate | (1) | 7.00 |
| | Volpo S-2 | Steareth-2 | (2) | 0.40 |
| | Volpo S-10 | Steareth-10 | (2) | 0.80 |
| | Pemulen TR-2 | Acrylate/C 10-30 Alkyl | (3) | 0.18 |
| | | Acrylate Crosspolymer | | |
| | Hetester PHA | Propylene Glycol | (4) | 5.00 |
| | | Isoceteth-3 Acetate | | |
| | Performa V 825 | Synthetic Wax | (5) | 0.80 |
| | Dow Corning 200 | Dimethicone | (6) | 1.00 |
| | (100 cs) | | | |
| | Oxynex K liquid | PEG-8 (and) Tocopherol | (1) | 0.10 |
| | | (and) Ascorbyl Palmitate | | |
| | | (and) Ascorbic Acid (and) | | |
| | | Citric Acid | | |
| B | Eusolex 232 | Phenylbenzimidazole | (1) | 1.00 |
| | | Sulfonic Acid | | |
| | Triethanolamine | Triethanolamine | (1) | 0.90 |
| | Propane-1,2-diol | Propylene Glycol | (1) | 2.00 |
| | Preservatives | | | q.s. |
| | Water, demineralised | Aqua | | to |
| | | | | 100.00 |

Preparation:

For the neutralisation of Eusolex 232, the triethanolamine is added to the water of phase B, and Eusolex 232 is added with stirring. After complete dissolution, the remaining raw materials of phase B are added and warmed to 80° C. Phase A is combined apart from the Pemulen and warmed to 80° C. The Pemulen is then stirred into phase A. Phase B is slowly added to phase A with stirring, and the mixture is homogenised and cooled with stirring.

Notes:

Preservatives:

0.05% of propyl 4-hydroxybenzoate

0.15% of methyl 4-hydroxybenzoate

Sources of Supply:

(1) Merck KGaA, Darmstadt

(2) Croda, Nettetal

(3) Goodrich, Neuss

(4) ROVI, Schlüchtern

(5) New Phase, N.J. 08554

(6) Dow Corning, Wiesbaden

1. Particles comprising an inorganic network and organic compounds covalently bonded to the network via a spacer group, characterised in that the organic compounds are present in the interior of the particles and optionally also on the surface of the particles.

2. Particles according to claim 1, characterised in that they are spherical particles.

3. Particles according to claim 1 , characterised in that the particles have a size of 1 nm to 250 μm.

4. Particles according to claim 1, characterised in that the inorganic network is an oxide, oxide hydrate, phosphate, carbonate, sulfate or nitride.

5. Particles according to claim 4, characterised in that the oxide is magnesium oxide, aluminium oxide, silicon oxide, zinc oxide, cerium oxide, titanium oxide, zirconium oxide, manganese oxide, boron oxide, iron oxide or a mixture thereof.

6. Particles according to claim 1, characterised in that the inorganic network is silicon oxide.

7. Particles according to claim 1, characterised in that the organic compounds covalently bonded to the network are selected from the group of light-absorbent organic compounds, compounds having antioxidant and/or free-radical-inhibiting properties, repellents, preservatives and/or derivatives of these compounds.

8. Particles according to claim 7, characterised in that the light-absorbent compound is selected from the group of dibenzoylmethane derivatives, aminobenzoic acid, cinnamic acid, salicylic acid, benzylidenecamphor, phenylbenzimidazole, diphenyl acrylate, triazine, benzophenone, diarylbutadiene, vinyl-containing amides and/or derivatives thereof.

9. Particles according to claim 7, characterised in that the compounds having antioxidant and/or free-radical-inhibiting properties are selected from flavonoids, coumaranones, vitamins, BHT and/or derivatives thereof.

10. Particles according to claim 7, characterised in that the repellents are selected from amides and/or derivatives thereof.

11. Particles according to claim 1, characterised in that the spacer group has the general formula
(-A)aMe(R1)b(R2)c-X-
where
A=O, S, NH,
Me=Si, Al, Ti, Zr,
R1 and R2 may be identical or different and denote a straight-chain or branched alkyl,
alkoxy, halogen or hydroxyl group
a is 1 to 3,
b and c may be 0 2, with the condition that a+b+c=3 for Me=Si, Ti, Zr and a+b+c=2 for Me=Al, and
X=a straight-chain or branched alkyl group having up to 20, preferably having 1 to 12 and particularly preferably having 3 to 12, carbon atoms, where the spacer group is bonded to the inorganic network via the substituent (A)a.

12. Particles according to claim 1, characterised in that the surface of the particles is additionally functionalised.

13. Particles according to claim 12, characterised in that organic compounds are covalently bonded to the surface of the particles.

14. Particles according to claim 13, characterised in that the organic compounds are selected from the group of light-absorbent organic compounds, compounds having antioxidant and/or free-radical-inhibiting properties, repellents, preservatives and/or derivatives of these compounds.

15. Particles according to claim 12, characterised in that the surface of the particles is functionalised with the same organic compound that is present in the interior of the particles and/or with organic compounds which are different therefrom.

16. Process for the preparation of particles according to claim 1, comprising
a) the reaction of an organic compound with formation of a covalent bond to a substance having at least two reactive groups separated by a spacer group and
b) the copolymerisation of the compound obtained in a) with a precursor which is suitable for the construction of an inorganic network.

17. Process according to claim 16, characterised in that the substance having at least two reactive groups separated by a spacer group has the general formula
(R3A)aMe(R1)b(R2)c-X-Y
where
A=O, S, NH
Me=Si, Al, Ti, Zr
R1 and R2 may be identical or different and denote a straight-chain or branched alkyl,
alkoxy, halogen or hydroxyl group
R3=hydrogen or a straight-chain or branched alkyl group
a=1 to 3
b and c may be 0 2, with the condition that a+b+c=3 for Me=Si, Ti, Zr and a+b+c=2 for Me=Al, and
X=a straight-chain or branched alkyl group having up to 20, preferably having 1 to
12 and particularly preferably having 3 to 12, carbon atoms, and
Y=straight-chain or branched alkenyl, alkynyl, alkoxy, SH, NH2, carboxyl, halogen or hydroxyl, where the covalent bond is formed between the organic compound and the spacer group with cleavage or via linking to the reactive group Y.

18. Process according to claim 16, characterised in that the organic compounds are selected from the group of light-absorbent organic compounds, compounds having antioxidant and/or free-radical-inhibiting properties, repellents, preservatives and/or derivatives of these compounds.

19. Process according to claim 16, characterised in that step b) is a hydrolytic polycondensation.

20. Process according to claim 19, characterised in that compounds of the elements magnesium, aluminium, silicon, zinc, cerium, titanium, zirconium, manganese, boron and iron and/or mixtures thereof, which contain hydroxides, halides, alkoxides or other hydrolysable groups, are employed as precursor.

21. Process according to claim 16, characterised in that the precursor is an alkoxide of silicon.

22. Process according to claim 16, characterised in that the surface of the particles is additionally functionalised.

23. Process according to claim 22, characterised in that organic compounds are covalently bonded to the surface of the particles.

24. Process according to claim 23, characterised in that the organic compounds are selected from the group of light-absorbent organic compounds, compounds having antioxidant and/or free-radical-inhibiting properties, repellents, preservatives and/or derivatives of these compounds.

25. Process according to claim 16, characterised in that the surface of the particles is additionally functionalised with the compounds obtained in a).

26. Use of particles according to claim 1 in formulations and compositions.

27. Compositions comprising particles according to claim 1 and at least one cosmetically, pharmaceutically and/or dermatologically tolerated carrier and/or adjuvant.

28. Compositions according to claim 27, characterised in that they are compositions having light-protection properties.

29. Compositions according to claim 27, characterised in that they are compositions which can be applied topically.

30. Compositions according to claim 1, characterised in that the compositions additionally comprise at least one organic and/or inorganic UV filter.

31. Compositions according to claim 1, characterised in that the compositions comprise at least one self-tanning agent.

32. Compositions according to claim 1, characterised in that the compositions comprise at least one photostabiliser.

33. Compositions according to claim 32, characterised in that the photostabiliser is selected from compounds conforming to the formula V 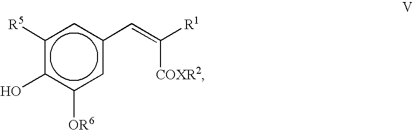
where
R1 is selected from C(O)CH3, CO2R3, C(O)NH2 and C(O)N(R4)2;
X is O or NH;
R2 stands for a linear or branched C1-30-alkyl radical;
R3 stands for a linear or branched C1-20-alkyl radical,
all R4, independently of one another, stand for H or linear or branched C1-8-alkyl radicals
R5 stands for H, a linear or branched C1-8-alkyl radical or a linear or branched O-C1-8-alkyl radical and
R6 stands for a C1-8-alkyl radical.

34. Compositions according to claim 1, characterised in that the compositions comprise one or more further UV filters which are selected from the group consisting of 3 (4′-methylbenzylidene)-dl-camphor, octyl methoxycinnamate, 3,3,5-trimethylcyclohexyl salicylate, 2 ethylhexyl 4 (dimethylamino)benzoate, 2 ethylhexyl 2 cyano-3,3-diphenylacrylate, 2 phenylbenzimidazole-5-sLllfonic acid and potassium, sodium and triethanolamine salts thereof.

35. Compositions according to claim 1, which are suitable for the protection of body cells against oxidative stress, characterised in that they preferably comprise one or more antioxidants.
